(19) United States
(12) Patent Application Publication
(10) Pub. No.: US 2025/0051863 A1
(43) Pub. Date: Feb. 13, 2025

(54) AUTOMATED MICROBIAL FLUID SAMPLING AND ANALYSIS SYSTEMS AND PROCESSES

(71) Applicant: Gate Scientific, Inc., Milpitas, CA (US)

(72) Inventors: Bradford Jensen TAFT, San Francisco, CA (US); Kristian Michael SCABOO, Castro Valley, CA (US); Morten Juel JENSEN, Saratoga, CA (US); Vincent CUEVAS, Pico Rivera, CA (US); Elhadj Sofiane HAMAMA, Oakland, CA (US)

(73) Assignee: Gate Scientific, Inc., Milpitas, CA (US)

(21) Appl. No.: 18/913,912

(22) Filed: Oct. 11, 2024

Related U.S. Application Data
(63) Continuation-in-part of application No. PCT/US2023/018203, filed on Apr. 11, 2023.
(60) Provisional application No. 63/362,947, filed on Apr. 13, 2022.
(60) Provisional application No. 63/362,950, filed on Apr. 13, 2022.

(51) Int. Cl. C12Q 1/70; B01L 3/00; B01L 7/00; C12Q 1/6851

(52) U.S. Cl. CPC C12Q 1/701; B01L 3/502761; B01L 7/52; C12Q 1/6851; B01L 2200/04; B01L 2200/0647; B01L 2200/16; B01L 2300/0816; B01L 2300/18

(57) ABSTRACT

Automated microbial fluid sampling and analysis systems and processes are disclosed herein. Such a system can include a processor that can execute stored computer instructions to control the air sampling and analysis system, a consumable cartridge including a cartridge body and a sample capture substrate (SCS), an air handler to move air through the SCS, a thermocycler to bring the air sampling reaction chamber to one or more desired temperatures to perform nucleic acid amplification directly on the SCS, and optics that can measure fluorescent signal from the air sampling reaction chamber. The system can automatically collect the sample, and can automatically perform the nucleic acid amplification and analysis.

### CROSS-REFERENCE TO RELATED APPLICATIONS

This application is a continuation-in-part of International Application No. PCT/US2023/018203, filed Apr. 11, 2023, which claims priority to U.S. Provisional Application No. 63/362,947, filed Apr. 13, 2022, and 63/362,950, filed Apr. 13, 2022, all of which are incorporated by reference herein in their entireties.

### BACKGROUND

Throughout the history of humankind, increasing cleanliness has led to improved health and greater lifespans. This has been the case with simple improvements such as better personal hygiene, as well as with more complicated improvements like the development of treated water and sewage systems.

The outbreak of SARS-CoV-2 emphatically demonstrated that further improvements to cleanliness were needed. Particularly, at present, there are only limited avenues for detecting and/or limiting airborne pathogens. Because of this inability to detect and/or limit airborne pathogens, many countries experienced massively detrimental lockdowns, illnesses and deaths.

While SARS-CoV-2 demonstrated shortcomings in detecting and treating airborne pathogens, similar shortcomings exist in other environments in which the target substance or pathogen is at low levels. Accordingly, improvements to the detection of target substances and/or pathogens, including airborne pathogens are desired.

### BRIEF SUMMARY

A system of one or more computers can perform particular operations or actions by virtue of having software, firmware, hardware, or a combination of them installed on the system that in operation causes or cause the system to perform the actions. One or more computer programs can perform particular operations or actions by virtue of including instructions that, when executed by data processing apparatus, cause the apparatus to perform the actions. One general aspect includes an air sampling and analysis system. The air also includes a processor that can execute stored computer instructions to control the air sampling and analysis system. The air also includes a consumable cartridge including a cartridge body and a sample capture substrate (SCS). The air also includes an air handler that can move air through the SCS. The air also includes a thermocycler that can bring the air sampling reaction chamber to one or more desired temperatures to perform nucleic acid amplification directly on the SCS. The air also includes optics that can measure fluorescent signal from the air sampling reaction chamber. Other embodiments of this aspect include corresponding computer systems, apparatus, and computer programs recorded on one or more computer storage devices, each can perform the actions of the methods.

Implementations may include one or more of the following features. The system further including a communications module for electronically transmitting data to another system and optionally for receiving operating instructions. The SCS extends across an air sampling reaction chamber defined by the cartridge body. The cartridge receiver includes at least one sealing member sealingly couplable to the cartridge body around the air sampling reaction chamber to thereby enclose the air sampling reaction chamber and SCS. The enclosed air sampling reaction chamber has volume of between 50 1 and 200 1. The enclosed air sampling reaction chamber has a volume of approximately 100 1. The cartridge body includes a front and a back, where the sealing member includes a first sealing member and a second sealing member, where the first sealing member can sealingly couple to the front of the cartridge body around the air sampling reaction chamber, and where the second sealing member can sealingly couple to the back of the cartridge body around the air sampling reaction chamber. Sealingly coupling the first sealing member to the front of the cartridge body and the second sealing member to the back of the cartridge body encloses the air sampling reaction chamber and the SCS. The first sealing member is transparent, where the cartridge receiver is positioned with respect to the optics to expose the SCS to the optics through the first sealing member. The system further including an adhesive can sealingly couple the sealing members to the cartridge body around the air sampling reaction chamber. The adhesive is heat activated. The adhesive extends around a perimeter of the sealing member. The system further including a sealer can seal the sealing member to the cartridge body. The system further including: a mechanism for ejecting spent cartridge bodies and positioning new cartridge bodies for sample capture and analysis; and a magazine including a plurality of new cartridge bodies and cartridge receivers. The cartridge body includes a first containment capsule including dry reagents and a second containment capsule including aqueous reagents. The cartridge body further includes a first fluidic channel coupling the first containment capsule and the second containment capsule, and a second fluidic channel coupling the first containment capsule and the air sampling reaction chamber. Each of the first containment capsule and the second containment capsule is divided by a membrane into a first chamber and a second chamber. The first chamber of the first containment capsule contains the dry reagents, where the first chamber of the second containment capsule contains the aqueous reagents, and where the second chamber of each of the first containment capsule and the second containment capsule contains at least one piercing feature that can pierce the membrane and release contents of the first chamber. The first fluidic channel and the second fluidic channel couple to the second chamber of the first containment capsule, and where the first fluidic channel couples to the second chamber of the second containment capsule. The system further including: a first actuator that can pierce the membrane of the first containment capsule; a second actuator that can compress the second containment capsule to pierce the membrane; and a third actuator to compress the first containment capsule to move the contents of the first containment capsule into the air sampling reaction chamber. The processor can: control the first and second actuators to mix the aqueous reagents and the dry reagents; and control the third actuator to move the mixed reagents through the second fluidic channel into the air sampling reaction chamber. The consumable cartridge includes one or more containment capsules containing nucleic acid amplification primers specific for at least one of Clostridium botulinum, listeria, campylobacter, trichinosis, Staphylococcus aureus including methicillin and vancomycin-resistant Staphylococcus aureus, Pseudomonas aeruginosa, Klebsiella pneumonia, Mycobacterium tuberculosis, Neisseria gonorrhoeae, enterococcus including vancomycin-resistant enterococcus, salmonella including fluoroquinolone-resistant salmonella, c. difficile including clindamycin-resistant c. difficile, Bacillus anthracis, a. baumannii including multidrug-resistant a. baumannii, Streptococcus pneumonia, Candida albicans, Pseudomonas aeruginosa, Acinetobacter baumannii, Stenotrophomonas maltophilia, e. coli including fluoroquinolone-resistant e. coli and e. coli ol57:h7, Legionella pneumophila, Streptococcus pyogenes, ebola virus, dengue virus, novavirus, viruses that cause lassa fever, yellow fever, marburg hemorrhagic fever and crimean-congo hemorrhagic fever, rhinovirus including types a, b and c, coronavirus including sars cov, sars cov-2 (including alpha (b.1.1.7 and q lineages), beta (b.1.351 and descendent lineages), gamma (p. 1 and descendent lineages), epsilon (b.1.427 and b.1.429), eta (b.1.525), iota (b.1.526), kappa (b.1.617.1 and 1.617.3), mu (b.1.621, b.1.621.1), zeta (p.2), delta (b.1.617.2 and ay lineages) and omicron (b.1.1.529 and ba lineages)) and mers, adenovirus, influenza virus including types a and b; parainfluenza virus; respiratory syncytial virus (including types a and b); enterovirus; norovirus (including genogroups gi, gii, giii, giv, gv, gvi, and gvii); rotavirus, astrovirus, viruses that cause measles, rubella, chickenpox/shingles, roseola, smallpox and fifth disease, chikungunya virus, hepatitis (including types a, b, c, d, and e), herpesvirus, papilloma virus; molluscum contagionsum; polio virus; rabies virus, viruses that cause viral meningitis and encephalitis, hln1,hln2,h2n2,h2n3,h3n1,h3n2,h3n8,h5n1,h5n2,h5n3,h5n6,h5n8,h5n9,h6n1,h6n2,h7n1,h7n2, h7n3,h7n4,h7n7,h7n9,h9n2,hl0n7,hl0n8,hlln2,hlln9,hl7n10,hl8nll,hpiv-1,hpiv-2, hpiv-3, hpiv-4, hadv-b, hadv-c, 229e, oc43, n163, hukl, sin nombre orthohantavirus, black creek canal orthohantavirus, puumala virus, thaland virus, hrv-a1, hrv-a2, hrv-a713, hrv-a15, hrv-a16, hrv-a1825, hrv-a2834, hrv-a36, hrv-a3841, hrv-a4347, hrv-a4951, hrv-a5368, hrv-a71, hrv-a7378, hrv-a8082, hrv-a85, hrv-a8890, hrv-a9496, hrv-a98, hrv-a100103, hrv-b36, hrv-bl4, hrv-bl 7, hrv-b26, hrv-b27, hrv-b35, hrv-b37, hrv-b42, hrv-b48, hrv-b52, hrv-b69, hrv-b70, hrv-b72, hrv-b79, hrv-b83, hrv-b84, hrv-b86, hrv-b9193, hrv-b97, hrv-b99, and hrv-cl-51, human associated microbes such as actinomyces, aerococcus, akkermansia, alistipes, alloiococcus, anaerococcus, anaerotruncus, atopobium, bacteroides, bamesiella, bifidobacterium, blautia, butyrivibrio, chlamydia, clostridium, corynebacterium, cutibacterium (formerly propionibacterium), dialister, dysgonomonas, enterobacter, enterococcus, escherichia, faecalibacterium, fusobacterium, gardnerella, gemella, haemophilus, klebsiella, kocuria, lactobacillus, lactococcus, megasphera, methanobrevibacter, micrococcus, mobiluncus, moraxella, mycobacterium, mycoplasma, neisseria, oxalobacter, papillibacter, parabacteriodes, parvimonas, peptoniphilus, peptostreptococcus, porphyromonas, prevotella, Pseudomonas, roseburia, ruminococcus, sneathia, spirochaeta, staphylococcus, streptococcus, villonella, altemaria, aspergillus, candida, cladosporium, curvularia, embellisia, fusarium, penicillium, saccharomyces, stachybotrys, thermomyces, trichophyton, malassezia, and rhodotorula, human mase-p, allergenic molds including altemaria, aspergillus (including a. fumigatus, a. versicolor and a. flavus), cladosporium, cryptococcus (including cryptococcus neoformans), Histoplasma capsulatum, stachybotrys (including stachybotrys chartarum), penicillium (including p. brevicompactum, p. chrysogenum, p. citrinum, p. corylophilum, p. cyclopium, p. expansum. p. fellutanum, p. spinulosum, and p. viridicatum), helminthosporum, epicoccum, fusarium (including f. solani, f. oxysporum, f. moniliforme), aureobasidium, phoma, smuts, rhizopus and mucor, and pollen including from plants and trees including ragweed, mountain cedar, ryegrass, maple, elm, mulberry, pecan, oak, pigweed/tumbleweed, arizona cypress, alder, ash, beech, birch, box elder, cedar, cottonwood, date palm, elm, mulberry, hickory, juniper, oak, pecan, phoenix palm, red maple, silver maple, sycamore, walnut, willow bermuda grass, johnson grass, kentucky bluegrass, orchard grass, rye grass, sweet vernal grass, timothy grass, english plantain, lamb's quarters, redroot pigweed, sagebrush and tumbleweed (russian thistle). The optics can measure at least four channels of fluorescent signal. The processor can control the air handler to move air through the SCS at a desired flow rate. The flow rate is between O. 1 lpm/mm2 of the SCS and 10 lpm/mm2 of the SCS. The system can collect and analyze between 10 and 80 air samples per day, and where at least some of those air samples are collected by moving between approximately 500 1 of air through the SCS and 100,000 1 of air through the SCS. The system can generate less than 50 db at a distance of 36 inches from the system at a flow rate between O. 1 lpm/mm2 and 10 lpm/mm2 of the SCS. The flow rate is approximately 1001 μm. The SCS has an area of between 0.5 mm2 and 2,000 mm2. The SCS has a thickness approximately between 0.02 mm and 0.5 mm. The air handler and SCS can achieve of pressure drop of between approximately 1 kpa and 30 kpa. The SCS has a weight of between approximately 5 g/m2 and approximately 60 g/m2. The SCS has at least one of: an area of approximately 115 mm2; and a width or diameter of approximately 12 mm. The system is capable of collecting and analyzing up to approximately 32 air samples per day, and where at least some of those air samples are collected by moving approximately 3,0001 of air through the SCS. The thermocycler can hold the reaction chamber at a temperature of between 20 c 60 c during a reverse transcription phase prior to a nucleic acid amplification phase. Lysis of analyte occurs during sample capture and a reverse transcription phase. Target nucleic acid becomes available for exposure to reagents during sample capture and a reverse transcription phase. The system performs rt-qpcr. Implementations of the described techniques may include hardware, a method or process, or computer software on a computer-accessible medium.

One general aspect includes a method of automated air sampling and analysis. The method of automated air sampling also includes passing air through a sample capture substrate (SCS) to capture analyte. The method of automated air sampling also includes enclosing the SCS within a reaction chamber. The method of automated air sampling also includes performing a nucleic acid amplification reaction directly on the SCS within the reaction chamber. The method of automated air sampling also includes measuring a fluorescent signal from the reaction chamber and from the SCS, where the method is performed inside a single instrument without human intervention. Other embodiments of this aspect include corresponding computer systems, apparatus, and computer programs recorded on one or more computer storage devices, each can perform the actions of the methods.

Implementations may include one or more of the following features. The method further including detecting presence of one or more airborne pathogens based on the measured fluorescent signal. The method further including transmitting results to another system. The results indicate the detected presence of the one or more airborne pathogens. The method further including detecting absence of one or more airborne pathogens based on the measured fluorescent signal. The method further including transmitting results to another system. Air is passed through the SCS at between approximately 1 lpm/mm2 of the SCS and 5 lpm/mm2 of the SCS. The method further including generating less than 50 db at a distance of 36 inches at a flowrate between 0.1 lpm/mm2 of the SCS and 10 lpm/mm2 of the SCS. The SCS is connected to a cartridge body, the cartridge body defining the reaction chamber. The reaction chamber defined by the cartridge body has a diameter of approximately between 1 mm and 50 mm. The SCS has a thickness approximately between 0.02 mm and 0.5 mm. The SCS has a weight between approximately 1 g/m2 and 100 g/m2. Enclosing the SCS within the reaction chamber includes: inserting the cartridge body at least partially into a cartridge receiver including a first and second sealing member; and sealing the sealing members around a perimeter of the reaction chamber defined by the cartridge body. The enclosed reaction chamber has a volume between approximately 50 1 and approximately 200 1. The enclosed reaction chamber has a volume of approximately 100 1. The SCS fills approximately 1 1 chamber volume/mm2 of SCS. The cartridge body includes a front and a back, where the sealing member includes a first sealing member and a second sealing member, and where sealing the sealing members around the perimeter of the reaction chamber defined by the cartridge body includes: sealing the first sealing member to the front of the cartridge body; and sealing the second sealing member to the back of the cartridge body. Performing the nucleic acid amplification reaction directly on the reaction chamber and on the SCS includes: filling the reaction chamber with nucleic acid amplification reagents; and thermocycling the reaction chamber and the SCS. Filling the reaction chamber with nucleic acid amplification reagents includes: mixing aqueous nucleic acid amplification reagents with dry nucleic acid amplification reagents; and filling the mixed nucleic acid amplification reagents into the reaction chamber. The dry nucleic acid amplification reagents are contained within a first containment capsule, where the aqueous nucleic acid amplification reagents are contained in a second containment capsule, where the first containment capsule and the second containment capsule are linked by a first fluidic channel, and where the first containment capsule and the reaction chamber are linked by a second fluidic channel. Mixing aqueous nucleic acid amplification reagents with dry nucleic acid amplification reagents includes compressing the second containment capsule to move at least some of the aqueous nucleic acid amplification reagents to the first containment capsule to mix with the dry nucleic acid amplification reagents, and where filling the mixed nucleic acid amplification reagents into the reaction chamber includes compressing the first containment capsule to move at least some of the mixed nucleic acid amplification reagents from the first containment capsule to the reaction chamber via the second fluidic channel. The method further including: ejecting the cartridge body after completion of the measuring of the fluorescent signal from the reaction chamber and from the SCS; retrieving a new cartridge body from a magazine containing a plurality of unused cartridge bodies; and positioning the new cartridge body to pass air through the SCS of the new cartridge body. The SCS has a total autofluorescence ofless than 50% of baseline fluorescence. The nucleic acid amplification reaction includes at least one of: qpcr, per, rt-per, rt-qpcr, digital per, an isothermal amplification process, immuno-pcr, and proximity ligation per. Analyzing each of those between 6 and 48 samples subsequent to collection of that sample includes: enclosing the SCS within the reaction chamber, performing the nucleic acid amplification reaction directly on the reaction chamber and on the SCS, and measuring the fluorescent signal from the reaction chamber and from the SCS. Each of the collected samples captures analyte from between approximately 500 1 of air through the SCS and approximately 5,000 1 of air through the SCS. Air sampling commences upon receiving a signal from another system, such signal being initiated in response to a carbon dioxide level inside a building where the system is situated rising above a threshold level. Where the SCS is not electrostatically charged. Implementations of the described techniques may include hardware, a method or process, or computer software on a computer-accessible medium.

One general aspect includes a system for air sampling. The system also includes a consumable cartridge, the consumable cartridge including: a cartridge body defining an air sampling reaction chamber, and a sample capture substrate coupled to the cartridge and extending across the air sampling reaction chamber. The system also includes a cartridge receiver defining a receptacle that can receive the sample capture substrate, where the cartridge receiver includes first and second sealing members that are sealingly couplable to the cartridge body around the air sampling reaction chamber to thereby enclose the air sampling reaction chamber, where the sample capture substrate is enclosed within the air sampling reaction chamber. Other embodiments of this aspect include corresponding computer systems, apparatus, and computer programs recorded on one or more computer storage devices, each can perform the actions of the methods.

Implementations may include one or more of the following features. The system can have the air sampling reaction chamber integrated into the cartridge body. Air sampling is performed during predetermined hours that correspond to times of human occupancy above a certain density of humans per square foot. The sample capture substrate is coupled to the cartridge body around a perimeter of the air sampling reaction chamber. The sample capture substrate can capture airborne microbes. The sample capture substrate includes at least one of: polypropylene; polytetrafluoroethylene (ptfe); and polycarbonate. The sample capture substrate includes melt-blown polypropylene. The sample capture substrate exhibits no autofluorescence or autofluorescence at level that does not interfere with nucleic acid amplification analysis that uses fluorescent probes for target identification. The sample capture substrate generates less than 5% of total system fluorescence. The sample capture substrate exhibits autofluorescence of less than 50% of baseline fluorescence. The sample capture substrate does not inhibit nucleic acid amplification reactions. The sample capture substrate is stable at temperatures between 4(c and 1O0(c. At least one sealing member is transparent. The at least one sealing member includes at least one of: polycarbonate, polyester, polyethylene terephthalate, glass, and polypropylene. At least one sealing member includes an extruded film. The at least one sealing member has a thickness of less than approximately 200 m. The at least one sealing member has a thickness of between approximately 50 m and approximately 500 m. The system further including an adhesive that can sealingly couple the sealing member to the cartridge body around the air sampling reaction chamber. The adhesive is heat activated. The adhesive extends around a perimeter of the sealing member. The cartridge body includes a first containment capsule including dry reagents and a second containment capsule including aqueous reagents. The cartridge body further includes a first fluidic channel coupling the first containment capsule and the second containment capsule, and a second fluidic channel coupling the first containment capsule and the air sampling reaction chamber. The each of the first containment capsule and the second containment capsule is divided by a membrane into a first chamber and a second chamber. The first chamber of the first containment capsule contains the dry reagents, where the first chamber of the second containment capsule contains the aqueous reagents, and where the second chamber of each of the first containment capsule and the second containment capsule contains at least one piercing feature that can pierce the membrane and allow contents of the first chamber to enter the second chamber. The first fluidic channel and the second fluidic channel couple to the second chamber of the first containment capsule, and where the first fluidic channel couples to the second chamber of the second containment capsule. The dry reagents include lyophilized nucleic acid amplification reagents, and where the aqueous reagents include aqueous nucleic acid amplification reagents. At least one of the reagents includes a surfactant. The surfactant includes at least one of: tween; np40; and triton x-100. The air sampling reaction chamber has a width of between 1 mm and 50 mm The air sampling reaction chamber is circular and has a diameter of between 1 mm and 50 mm. The air sampling reaction chamber when enclosed by the first and second sealing members has volume of between 10 1 and 250 1. The enclosed air sampling reaction chamber has a volume of approximately 100 1. The system performs rt-qpcr. In some embodiments, the SCS is not electrically electrostatically charged. Implementations of the described techniques may include hardware, a method or process, or computer software on a computer-accessible medium.

One general aspect includes that the consumable sampling unit also includes a first portion defining a sample capture substrate (SCS). The consumable sampling unit also includes a second portion defining a receptacle that can receive the SCS and enclose it into a reaction chamber, where the SCS is enclosed within the reaction chamber. The consumable sampling unit also includes at least one containment capsule including at least nucleic acid amplification reagents, where the at least one containment capsule is fluidly couplable with the reaction chamber and the SCS. Other embodiments of this aspect include corresponding computer systems, apparatus, and computer programs recorded on one or more computer storage devices, each can perform the actions of the methods.

Implementations may include one or more of the following features. The consumable sampling unit where the at least one containment capsule includes: at least one first containment capsule containing dry nucleic acid amplification reagents; and at least one second containment capsule containing aqueous nucleic acid amplification reagents. The at least one containment capsule is located on the first portion. The at least one containment capsule is located on the second portion. The at least one first containment capsule is located on one of the first portion and the second portion, and where the at least one second containment capsule is located on the other of the first portion and the second portion. The at least one first containment capsule is fluidly connected to the at least one second containment capsule and to the reaction chamber. The first portion and the second portion are connected via a pivot. The first portion and the second portion are coupled. The first portion and the second portion are not connected. The SCS is not electrostatically charged. The sampling unit can collect a sample from air. The sampling unit can collect a sample from a liquid. The consumable sampling unit where the SCS includes at least one of: a membrane; a swab; a brush; and a scoop. The chamber includes a reaction chamber. The consumable sampling unit further including a second portion defining a receptacle. The receptacle can enclose the chamber of the first portion. The receptacle can receive at least the chamber and the SCS. The at least one containment capsule is located on the first portion. The at least one containment capsule is located on the second portion. The at least one containment capsule includes a first containment capsule including dry reagents and a second containment capsule including aqueous reagents. The consumable sampling unit further including a first fluidic channel coupling the first containment capsule and the second containment capsule, and a second fluidic channel coupling the first containment capsule and the air sampling reaction chamber. The each of the first containment capsule and the second containment capsule is divided by a membrane into a first chamber and a second chamber. The first chamber of the first containment capsule contains the dry reagents, where the first chamber of the second containment capsule contains the aqueous reagents, and where the second chamber of each of the first containment capsule and the second containment capsule contains at least one piercing feature that can pierce the membrane and allow contents of the first chamber to enter the second chamber. The first fluidic channel and the second fluidic channel couple to the second chamber of the first containment capsule, and where the first fluidic channel couples to the second chamber of the second containment capsule. The dry reagents include lyophilized nucleic acid amplification reagents, and where the aqueous reagents include aqueous nucleic acid amplification reagents. At least one of the reagents includes a surfactant. The surfactant includes at least one of: tween; np40; and triton x-100. Where the SCS is not electrically electrostatically charged. Implementations of the described techniques may include hardware, a method or process, or computer software on a computer-accessible medium.

Implementations may include one or more of the following features. The consumable sampling unit where the SCS includes at least one of: a membrane; a swab; a brush; and a scoop. The chamber includes a reaction chamber. The consumable sampling unit can further include a second portion defining a receptacle. The receptacle can enclose the chamber of the first portion. The receptacle can receive at least the chamber and the SCS. The at least one containment capsule is located on the first portion. The at least one containmentcapsule is located on the second portion. The at least one containment capsule includes a first containment capsule including dry reagents and a second containment capsule including aqueous reagents. The consumable sampling unit further including a first fluidic channel coupling the first containment capsule and the second containment capsule, and a second fluidic channel coupling the first containment capsule and the air sampling reaction chamber. The each of the first containment capsule and the second containment capsule is divided by a membrane into a first chamber and a second chamber. The first chamber of the first containment capsule contains the dry reagents, where the first chamber of the second containment capsule contains the aqueous reagents, and where the second chamber of each of the first containment capsule and the second containment capsule contains at least one piercing feature that can pierce the membrane and allow contents of the first chamber to enter the second chamber. The first fluidic channel and the second fluidic channel couple to the second chamber of the first containment capsule, and where the first fluidic channel couples to the second chamber of the second containment capsule. The dry reagents include lyophilized nucleic acid amplification reagents, and where the aqueous reagents include aqueous nucleic acid amplification reagents. At least one of the reagents includes a surfactant. The surfactant includes at least one of: tween; np40; and triton x-100. Where the SCS is not electrically electrostatically charged. Implementations of the described techniques may include hardware, a method or process, or computer software on a computer-accessible medium.

Implementations may include one or more of the following features. The reaction chamber may include a region with one or more capture probes that are bound to a surface and in contact with the reaction solution during the nucleic acid amplification. The capture probes may be bound to a region of the surface comprising the reaction chamber or bound to a surface surrounded by the reaction chamber. The surface may be the SCS. The surface may be a bead or beads within the reaction. The capture probes may be bound to an optically clear surface. The capture probes may hybridize with a solution phase reporter nucleic acid that is indicative of the presence of a target species captured on the SCS. The amount of reporter nucleic acid hybridized to the capture probes may be proportional or inversely proportional to the amount of target species captured on the SCS. The capture probes may be in contact with the solution during the amplification reaction. There may be multiple regions with different capture probe sequences on the surface that can hybridize to specific associated reporter nucleic acids indicating the presence or absence of multiple target species captured on the SCS. There may be multiple regions with similar capture probe sequences on the surface that can hybridize to specific associated reporter nucleic acids indicating the presence or absence of multiple target species captured on the SCS thereby implementing redundancy. The multiple regions of capture probe sequence may be spatially arranged on the surface in a microarray. A bead surface may have one or multiple capture probe sequences.

The hybridization of the reporter nucleic acid with the capture probes may cause a change in a detectable signal from the surface. The detectable signal may be fluorescent, chemiluminescent, optically absorbent or transmissive or refractive, electrochemical, electrical, capacitive, or gravimetric. The change in the signal may be positive or negative. The signal may be monitored during the course of the reaction or at the end of the reaction. The signal may be monitored at different temperatures. The signal may be monitored at each cycle or a subset of cycles of a PCR reaction. The signal may be monitored during the enzymatic extension step of a PCR reaction. The first PCR cycle demonstrating a detectable change may be related to the amount of target species captured on the SCS. The signal from the hybridization events of multiple capture probe sequences may be monitored to detect the presence or amount of multiple target species captured on the SCS. The signal from the hybridization events of multiple capture probe sequences may be monitored simultaneously.

Implementations may include one or more of the following features. The system may contain a camera to detect optical signal from the reaction chamber. The camera may detect the signal from the bulk solution within the reaction chamber. The camera may detect the signal change from one or more surface bound capture probes. The camera may detect the signal change from an array of capture probes of different sequences. The camera may detect anomalies in the fluidic filling of the reaction chamber. The camera may detect incomplete fluidic filling of the chamber. The camera may detect bubbles within the reaction chamber. The data from the camera may be used to correct for signal variations from the fluidic filling anomalies. The data from the camera may be used such that only regions of capture probes that are not affected by fluidic or other errors are used for determining bound capture probes. The data from the camera may be used to determine pathogen concentration on the SCS. The pathogen concentration on the SCS may be proportional to the pathogen concentration in the sampled air or fluid. The data from the camera may be used to determine an operation error in the function of the system. The error may be in the fluidic function. The functional error may cause an invalid test result. The system may contain a camera that reads a unique identifier on the consumable cartridge. The identifier may be a barcode such as a QR code or any other barcode format. The system may contain a camera that determines the position of the consumable cartridge by determining the location of a cartridge feature or the location of a barcode or the location of a mark on the cartridge or a label. The camera that determines the location of the consumable cartridge may be the same as the camera that reads the unique identifier or the camera that detects the signal from the PCR reaction. There may be one or multiple cameras in the system that determines the signal from the PCR reaction and the unique identifier on the consumable cartridge and the location of the cartridge.

The system may contain sensors or may be directly or indirectly connected with external sensors that, upon sensing an analyte, initiate the air or fluid sampling to start. The analytes may be particles, gasses, or biomolecules from humans, animals or the environment. The sensor may detect particles or another analyte that is indicative of the presence of occupants in the space being monitored by the system. The system may start the air sampling upon indication of the presence of occupants within the space being monitored. Example of sensors that can be used to sense an analyte to determine initiation of a test may be Particle Counting, Fluorescent Bio-Aerosol sensor, Mass-Spectrometer, Gas chromatograph-mass spectrometer, Gas chromatograph, Gas chromatograph-Ion Mobility, Ion Mobility, CO2 sensor, Volatile Organic Compound sensor, Ultrasonic occupancy sensor, Infrared occupancy sensor, Image sensor for particle sensing, Image sensor for occupant determination, Temperature sensor, Humidity sensor, Fluorescent sensor, Absorbance sensor, Electrochemical sensor, pH sensor, conductivity sensor, Raman sensor, FTIR (Fourier Transform Infrared Spectroscopy) sensor or any other type of sensor.

The air handler unit of the system may be designed to minimize dust and particulate accumulation, significantly reducing the risk of contamination and false positives during sample collection. The air handling chimney may feature smooth, sloped surfaces, avoiding sharp angles that may trap dust or particles, this design prevents contaminants from settling in corners or crevices, ensuring that no buildup occurs that may compromise sample accuracy if collected outside the intended sampling period. The system may contain a pre-filter to prevent system clogging and amplification inhibitors from reaching the SDS.

In addition to the geometric design, the unit may be constructed from materials with low surface energy, antistatic properties, and/or dust-repellent characteristics. These materials minimize particle adhesion and reduce contamination risk. Smooth surface finishes and optional dust-repellent coatings further enhance the device's ability to maintain a contaminant-free environment. Additional surface treatments may also be applied to inhibit the growth of mold, bacteria, and other microorganisms that may impact sample collection.

To prevent cross-contamination between sample collection events, the air sampler may be equipped with a closure mechanism that isolates the air handler unit from the surrounding environment. This system may include a complete closure or a filtration barrier to block external contaminants. The device may first conduct a blank run, during which a filter separates the air handler from the environment, effectively cleaning the air handling system. After a cleaning process or at any other time, an optional subsequent run may be performed to monitor for any residual contamination. If contamination is detected, it may be logged in a database and either used to notify the user for further cleaning or replacement, or kept as part of the analysis for the next sample collection, ensuring the results account for any prior contamination.

For sterilization, the system may employ ultraviolet (UV) light or heat-based sterilization methods to maintain the cleanliness of the air handler unit between sample collections. These methods ensure thorough disinfection without the need for manual intervention.

In addition, the system may include an auxiliary cleaning unit that allows for the use of liquid reagents for more intensive cleaning. This system may utilize reagents such as ethanol, isopropanol, distilled water, wash buffers, chlorine based cleaning solution, or any other solution that may reduce contamination. This additional unit ensures that the air handler system can be deeply cleaned, extending its operational lifespan and ensuring consistent sample integrity.

The consumable cartridge may be fabricated as individual units that each can perform a test or the consumable cartridge may be manufactured in sets that can perform 2 or 3 or 5 or 10 or 100 or any number of tests. Multiple consumable cartridges may be combined into a set such that one cartridge feeds or pulls the next one into the system. A construction method may be used for the consumable cartridge where the consumable features for a test is stamped or compression molded or cut into or out of a roll of material such that the consumable cartridge(s) can be rolled up. Manufacturing of the consumable cartridge may involve laminating various materials together using heat or pressure or a laser or light or ultrasonic energy, this would enable low cost per test. Consumable cartridges may be snapped together to form. Consumable cartridges may form a belt or line or disk or drum or a roll individually or when combined.

### BRIEF DESCRIPTION OF THE DRAWINGS

FIG. 1 is a schematic depiction of one embodiment of environment control system.

FIG. 2 is a schematic depiction of one embodiment of the fluid sampling and analysis system.

FIG. 3 is a depiction of one embodiment of the sampling and analysis system.

FIG. 4 is a perspective view of one embodiment of the sampling unit.

FIG. 5 is a perspective view of one embodiment of cartridge body partially inserted into a cartridge receiver.

FIG. 6 is an exploded view of one embodiment of the sampling unit.

FIG. 7 is a perspective front view of another embodiment of the sampling unit.

FIG. 8 is a perspective front view of another embodiment of the sampling unit in which that cartridge body is partially inserted into the cartridge receiver.

FIG. 9 is a front view the other embodiment of the sampling unit.

FIG. 10 is a back view of the other embodiment of the sampling unit

FIG. 11 is a perspective view of one embodiment of a transparent cartridge body.

FIG. 12 is a perspective section view of one embodiment of the cartridge body.

FIG. 13 is a side section view of the embodiment of the cartridge body.

FIG. 14 is an exploded perspective view of one embodiment of the sampling unit.

FIG. 15 is a front view of one embodiment of the sampling unit.

FIG. 16 is perspective view of another embodiment of a sampling unit.

FIG. 17 is a perspective view of another embodiment of the sampling unit with the SCS in the retainer.

FIG. 18 is a perspective view of a clamshell embodiment of the sampling unit in an open configuration.

FIG. 19 is a perspective view of a clamshell embodiment of the sampling unit in a closed configuration.

FIG. 20 is a perspective view of a flag embodiment of the sampling unit in an open configuration.

FIG. 21 is a perspective view of a flag embodiment of the sampling unit in a closed configuration.

FIG. 22 is a perspective view of a sampling unit including a sleeve-shaped cartridge receiver.

FIG. 23 is a perspective view of a sampling unit including bendable sealing members in an open configuration.

FIG. 24 is a perspective view of a sampling unit including bendable sealing members in a closed configuration.

FIG. 25A-FIG. 25E are a schematic depiction of one embodiment of operating a sampling unit.

FIG. 26 is a flowchart illustrating one embodiment of a process for collecting and analyzing a sample.

FIG. 27 is a flowchart illustrating one embodiment of a process for performing a nucleic acid amplification process.

FIG. 28A-FIG. 28B show two graphs, each depicting SARS-CoV-2 RT-qPCR curves from air and nasal swab samples.

FIG. 29 shows a graph depicting RT-qPCR curves for a positive control and for two SCS materials.

FIG. 30 shows a graph depicting RT-qPCR curves evaluating capture efficiency of SCS materials.

FIG. 31 shows capture and RT-qPCR amplification of aerosolized coronavirus using a cartridge body that includes a reaction chamber across which an SCS is connected.

### DETAILED DESCRIPTION

Detection of a target within a fluid can be challenging. This is particularly the case when the concentration of the target is low or when the target is of small size or is otherwise difficult to capture and/or separate from the fluid.

These challenges are particularly acute when the target is biological. The challenges arise as wet biochemical reactions can be utilized in the detection of such biological analytes. While such assay are routinely performed on materials such as blood, urine, skin, and saliva, detecting one or multiple target analyte in other fluids such as in water, sewage, or air presents unique challenges. These challenges can relate to, for example, turnaround time, sensitivity, inhibition, and automation.

Detecting of a target analyte, such as detecting airborne pathogens in built environments occupied by humans, is most useful when a fast turnaround time occurs between completion of taking of a sample and generating a result. When samples are taken off-site for analysis, the turnaround time can be hours or even days. To maximize usefulness of the results, an analysis should provide results fast enough to allow measures to be implemented to mitigate the effects of the detected target analyte. Such mitigating measures can include, for example, modifying ventilation parameters, controlling opening of windows, and/or evacuating the area and/or building containing the pathogen.

Target analyte, such as a pathogen, and specifically an airborne pathogen, can cause an infection and/or allergic reaction even when present in very low concentrations. Further, collecting a sampled from a large volume of fluid, such as by, for example, pumping massive volumes of air though a filter is not an adequate solution. For example, increasing the volume of air through a filter increases noise generated by the pumps and/or blowers driving the fluid through the filter. Further, increasing the flow rate of air through a filter also leads to increased noise generated by the air passing through the filter. Additionally, increasing flow rate through a filter increases a pressure drop across the filter, requiring more expensive equipment and greater energy consumption. Finally, many materials capable of capturing biological pathogens, such as a biological airborne pathogens, have high resistance to airflow. This high resistance to airflow leads to a greater pressure drop across the filter and greater noise generation. As a result of this, a sampling and analysis system must be sufficiently sensitive to thereby limit the volume of fluid passed through the filter to generate a sample.

Unlike tissue samples such as blood, many fluids, such as air including air in buildings, sewage, or treated and/or untreated water, does not contain a consistent type or concentration of compounds. For example, air contains particulates of widely varying sizes from sub micrometer to visible to the human eye. These particulates are made of widely varying chemical composition based on building type, outdoor air conditions, building use, weather, geography and many other factors. These compounds may inhibit a nucleic acid amplification analysis, for example, may inhibit Polymerase Chain Reaction (“PCR”) analysis. Limiting inhibition, including inhibition caused by the sample, can beneficially improve the accuracy, speed, and/or effectiveness of analysis of a collected sample.

To be useful, a testing system should be able to quickly gather and analyze samples. This can include gathering and analyzing a large number of samples over a time period, such as over all or portions of a day. Diagnostic testing of air has traditionally been a manual process, which does not lend itself to the rapid gathering and analyzing of samples. In some embodiments, a sampling and analysis system can be automated, and specifically can gather and analyze samples via automation. Such a device can, for example, be programmed and/or controlled according to stored computer code, to perform assays at any desired interval and send results electronically a control system such as a building control system.

### Definitions

As used herein, a “nucleic acid amplification process” is any process via which one or multiple nucleic acids are amplified. This can include a nucleic acid amplification process such as an isothermal amplification or a non-isothermal amplification such as polymerase chain reaction (PCR). Specific examples include qPCR, RT-qPCR, PCR, RT-PCR, loop mediated amplification (LAMP), recombinase polymerase amplification (RPA), transcription mediated amplification (TMA), helicase dependent amplification (HDA), nucleic acid amplification based amplification (NASBA), rolling circle amplification (RCA), catalytic hairpin assembly (CHA), hybridization chain reaction (HCR), strand displacement amplification (SDA) and exponential amplification reaction (EXPAR).

As used herein, a “cartridge” is physical component for use in connection with the sampling system and that includes a Sample Capture Substrate (“SCS”). The cartridge can comprise a variety of shapes and sizes, and can be made from a variety of materials. The cartridge can include a cartridge body that can define a reaction chamber, also referred to herein as an “air sampling reaction chamber.” The SCS can extend across the reaction chamber. The cartridge body can further include reagents for a nucleic acid amplification process. These reagents can include dry reagents (lyophilized reagents) and/or aqueous reagents. In some embodiments, these reagents can include one or multiple surfactants, probes such as fluorescent probes, reverse transcriptase, DNA polymerase, oligonucleotides or the like. A cartridge is also referred to herein as a “sampling unit”. In some embodiments the cartridge is consumable and is used only once to analyze one sample that is captured by the SCS that is part of the cartridge.

The cartridge can be used in connection with a fluid sampling and analysis system, or can be used without such a sampling and analysis system In some embodiments, the cartridge cab be used for collecting and analyzing a sample, such as a fluid. In some embodiments, the cartridge can be used to collect samples from a fluid such as air, water, sewage, a bodily fluid such as, for example, blood, spit, urine, semen, mucus, bile, a blood component, pus, or the like.

As used herein, a “cartridge body” can be a portion of the cartridge that can hold the SCS. Specifically, the cartridge body can define the reaction chamber across which the SCS can extend. Further, the cartridge body can contain one or multiple reagents for performing the nucleic acid amplification process. These one or multiple reagents can be contained within one or multiple containment capsules formed in the cartridge body. The cartridge body can be made of a variety of materials such as polypropylene, polycarbonate, or the like.

As used herein, a “Sample Capture Substrate”, also referred to herein as an “SCS” is a substrate that can capture and/or hold a sample. In some embodiments, the SCS can comprise a substrate through which a liquid or gaseous medium can flow and that captures analyte from that liquid or gaseous medium In embodiments in which the medium is a liquid, the liquid can be, for example, water, sewage, a bodily fluid, or the like. The gaseous medium can, in some embodiments include air such as from a room, a building, a vehicle such as a passenger compartment of a vehicle such as a bus, a train, an airplane, a portion of a building, or the like. The SCS is also referred to interchangeably herein as a filter membrane or as a filter.

The SCS can be made from a variety of materials including, for example, polypropylene including melt-blown polypropylene, polytetrafluoroethylene (PTFE), and polycarbonate. The SCS can be configured to be nucleic acid amplification compatible. This can include being stable at temperatures used in the nucleic acid amplification process, and/or not inhibiting the nucleic acid amplification processes. The SCS can further have no or limited autofluorescence. This can include generating less than a desired percent of total system fluorescence, such as, for example, less than 5% of total system fluorescence in a qPCR reaction.

As used herein, a “cartridge receiver” can be a component configured to matingly engage with the cartridge. The cartridge receiver can comprise a variety of shapes and sizes, and can be made from a variety of materials. The cartridge receiver can comprise a body made of, for example, polycarbonate, and specifically, molded polycarbonate. The cartridge receiver can further comprise one or more sealing members configured to be sealed to and around the reaction chamber of the cartridge body.

As used herein, a “sealing member” can be a component configured for sealing to the cartridge body and around the reaction chamber to thereby enclose a side of the reaction chamber. The sealing member can comprise a variety of shapes and sizes and can be made of a variety of materials including, for example, glass, polycarbonate, polypropylene, polyester, polyethylene terephthalate, or the like. The sealing member can comprise a film, and can, in some embodiments, comprises an extruded film The sealing member can comprise, for example, a polycarbonate film, a polyester film, a polyethylene terephthalate film, or a polypropylene film.

A sealing member can be sealed to the cartridge body after air has been flowed through the SCS. The sealing member can be sealed to the cartridge body to thereby enclose a side of the reaction chamber for performing the nucleic acid amplification process. In embodiments in which the reaction chamber has two open sides, a sealing member can be sealed to each side of the cartridge body around the reaction chamber to thereby enclose the reaction chamber and enclose the SCS within the reaction chamber. Together, the combination of the two sealing members, each sealing one of the open sides of the reaction chamber, can enclose the reaction chamber.

As used herein, a “containment capsule” comprises a region configured to hold material for performing the nucleic acid amplification process. This material can be one or multiple reagents including one or multiple aqueous reagents and/or one or multiple dry reagents. A containment capsule can be sealed, and can be, for example, a blister pack.

As used herein, “baseline fluorescence” is the total fluorescence measured before the nucleic acid amplification process from the reaction chamber when the reaction chamber is filled with reagents but does not include the SCS.

As used herein, “total system fluorescence” is the complete fluorescence of the reaction chamber when the reaction chamber is filled with reagents, the SCS, and a sample, and a nucleic acid amplification process has been completed, resulting in detection of one or more target analytes.

As used herein, a “fluidic channel” is a pathway through the cartridge body coupled to at least one chamber and through which a fluid can flow. A fluidic channel can be used to move reagents from one location in the cartridge body to another location in the cartridge body. For example, a fluidic channel can couple one or several chambers storing reagents to each other, and/or a fluidic channel can couple one or several chambers storing reagents to a reaction chamber.

As used herein, a “fluorescence channel” is a fluorescent wavelength or a range of fluorescent wavelengths. The fluorescence channel can be associated with one or multiple fluorescent probes that bind to that target analyte. In some embodiments an optical unit can emit and/or detect multiple fluorescent channels corresponding to multiple fluorescent probes, each of which recognizes and anneals to nucleic acids of distinct sequence.

As used herein, a “sampling module” is a portion of a sampling and analysis system that is configured facilitate in collecting a sample with the cartridge, and specifically with the cartridge body. The sampling module can, in some embodiments, be configured to move fluid through a reaction chamber of a cartridge body, which fluid can be air. The sampling module can comprise one or multiple pumps, vacuums, fans, impellers, or the like. The sampling module is also referred to herein as an “air handler” and a “fluid transport module”.

As used herein, a “thermocycler” is a device configured to hold at a desired temperature for the purpose of bringing one or more adjacent components in close physical contact such as a reaction chamber to the same desired temperature. A thermocycler can also cyclically heat and cool one or more adjacent components, such as to perform a PCR reaction.

### Sampling and Analysis System

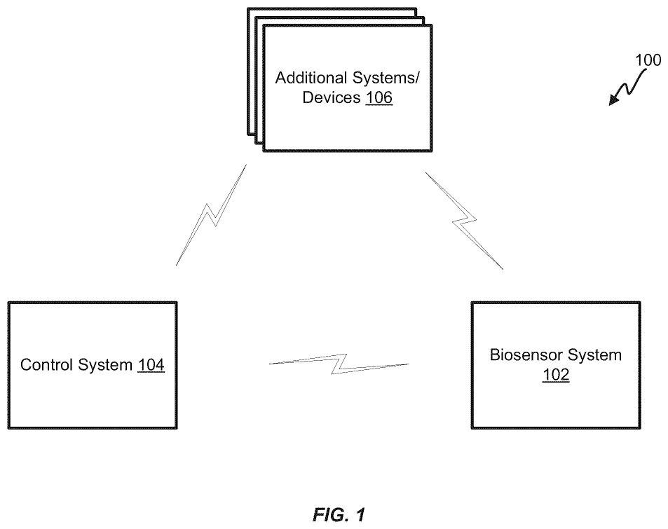

With reference now to FIG. 1, a schematic depiction of one embodiment of environment control system 100 is shown. The environment control system 100 can be configured to sample and analyze a fluid, such as air, water, sewage, or the like. The environment control system 100 can include a fluid sampling and analysis system 102 (also referred to herein as “sampling and analysis system 102” or as a “biosensor system 102”, a control system 104, and/or one or several additional systems/devices 106.

The sampling and analysis system 102, which will be discussed in greater detail below, can be configured to gather and analyze one or multiple fluid samples. Specifically, this can include gathering and analyzing one or multiple air samples within a building. The sampling and analysis system 102 can be communicatingly connected with a control system 104. The control system 104 can be a distinct device, such as a control unit from which operating instructions are sent to the biosensor system 102. The control system can send operating instructions to the biosensor manually using human intervention or according to pre-programmed instructions, including instructions such as changing sampling frequency or cartridge selection in response to previous results transmitted to the control system 104 by the biosensor system 102. Alternatively the control system can be user interface through which a user can view collected data.

In some embodiments, the control system 104 can comprise software enabling a user to log into the control system 104. In some embodiments, the user can remotely log-in to the control system 104, or can, in-person, log-in to the control system 104. In some embodiments, via logging-in to the control system 104, the user analyze data and/or transmit operating instructions via, for example, a web browser interface or the like. Additional systems/devices can be any system or device that performs a function in response to data received from the biosensor system 102 and/or any system or device that transmits date to the biosensor system 102, such as a control system, an access portal, an alarm and a notification system. Additionally, see U.S. patent application Ser. Nos. 17/094,632 and 17/180,627, hereby incorporated by reference, for examples of biosensors communicating with additional systems such as building ventilation systems. In some embodiments the biosensor system 102 can be programmed to send instructions and other data directly to additional systems/devices 106.

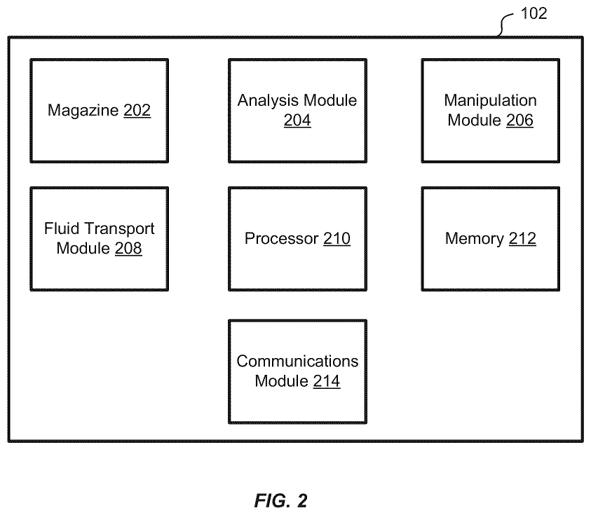

With reference now to FIG. 2, a schematic depiction of one embodiment of the fluid sampling and analysis system 102 is shown. The sampling and analysis system 102 can include a magazine 202. The magazine 202 can be configured to hold one or multiple sampling units, which sampling units can include, for example, a cartridge body and a cartridge receiver. In some embodiments, each of the sampling units can comprise a disposable sampling unit that can be used for the collection and analysis of one sample. The magazine 202 can be configured to hold one or multiple sampling units in one or multiple desired positions and/or in one or multiple desired orientations. In some embodiments, the magazine 202 can be accessed to load one or multiple new sampling units into the magazine 202 and/or can be accessed to remove one or multiple spent sampling units from the magazine 202. In some embodiments, one or multiple sampling units can be removed from magazine 202, and can be used to collect and analyze a sample.

The sampling and analysis system 102 can include an analysis module 204. The analysis module 204 can be configured to analyze the sample in one or multiple sampling units. In some embodiments, the analysis module 204 can be configured to perform a nucleic acid amplification and to analyze the output of that nucleic acid amplification to determine the presence and/or concentration of one or multiple target analytes. The nucleic acid amplification can be, for example, an isothermal amplification process or a non-isothermal amplification process. In some embodiments, for example, the isothermal amplification process can include, for example, one or multiple of: Loop mediated amplification (LAMP); Recombinase polymerase amplification (RPA); Transcription mediated amplification (TMA); Helicase dependent amplification (HDA); Nucleic acid amplification based amplification (NASBA); Rolling Circle Amplification (RCA); Catalytic Hairpin Assembly (CHA); Hybridization Chain Reaction (HCR); Strand Displacement Amplification (SDA); Exponential Amplification Reaction (EXPAR); or the like. In some embodiments, the non-isothermal amplification process can include at least one of, for example, qPCR, PCR, RT-qPCR, digital PCR, immuno-PCR, proximity ligation PCR, or the like.

In embodiments in which the analysis module 204 is configured to perform the non-isothermal amplification process, the analysis module 204 can include, for example, a thermocycler. In some embodiments, the thermocycler can be configured to cyclically vary a temperature of the one or multiple sampling units containing a sample being analyzed. In some embodiments, the varying of the temperature can include the cyclic heating and/or cooling of the one or multiple sampling units containing the sample being analyzed.

In some embodiments, the analysis module 204 can further include optics. In some embodiments, the optics can be configured to emit and receive electromagnetic energy from the sample and to detect the presence or absence of one or multiple of the target analytes based on that received electromagnetic energy. In some embodiments, the received electromagnetic energy can comprise light emitted by the sample, and specifically by, for example, probes bound to one or multiple of the target analytes. This light emitted by the sample can comprise fluorescence.

In some embodiments, each of the target analytes can be identified with one or multiple probes that together form a unique fluorescence profile. The optics can be configured to emit and receive the fluorescence and detect the presence or absence of one or multiple of the target analytes based on the fluorescence profile and/or fluorescence profiles received from the sample. In some embodiments, the optics can be multichannel to enable the simultaneous detection of multiple of the target analytes.

The sampling and analysis system 102 can include a manipulation module 206. The manipulation module 206 can be configured to move sample units to and/or between positions within the sampling and analysis system 102. In some embodiments, the manipulation module 206 can comprise a mechanism for ejecting spent cartridge bodies and positioning new cartridge bodies for sample capture and analysis.

In some embodiments, the manipulation module 206 can be configured to remove one or multiple sampling units from the magazine 202 and position the one or multiple sampling units to collect a sample. After the sample has been collected, the manipulation module 206 is configured to move the one or multiple sampling units from the position to collect the sample to the analysis module 204. In some embodiments, the manipulation module 206 can be further configured to manipulate one or multiple features of the one or multiple sampling units to facilitate the performing of the analysis. This can include, for example, manipulating one or multiple containment capsules containing one or multiple reagents to cause the mixing of those reagents and the entrance of those reagents into the reaction chamber of the sampling unit having those containment capsules. In some embodiments, after the analysis has been completed, the manipulation module 206 can be configured to remove the one or multiple sampling units from the analysis module 204 and to dispose the one or multiple sampling units.

In some embodiments, the sampling and analysis system 102 can include a fluid transport module 208, also referred to herein as a “sampling module 208” or as “air handler 208”. The sampling module 208 can be configured to direct and/or draw fluid through the one or multiple sampling units to thereby collect a sample on a portion of the one or multiple sampling units. In some embodiments, the sampling module 208 can comprise, for example, one or multiple fans, pumps, vacuums, or the like. The sampling module 208 can, in some embodiments, be configured to push fluid through the one or multiple sampling units, and/or to draw or pull fluid through the one or multiple sampling units. In some embodiments, the sampling module 208 can comprise a portion directing already flowing air, such as air being moved through an HVAC vent through the sampling unit. In such an embodiment, the sampling module 208 is configured to direct fluid through the one or more sampling units.

In some embodiments, the sampling and analysis system 102 can include one or multiple processors 210. In some embodiments, the processor 210 can be configured to generate signals to control operation of the other modules of the sampling an analysis system 102, and/or receive signals from the other modules of the sampling an analysis system 102. In some embodiments, for example, the processor 210 can receive data from the magazine 202 indicating a number of sampling units in the magazine 202 and/or whether the magazine 202 should be refilled. In some embodiments, the processor 210 can receive data from the analysis module 204 indicating, for example, a temperature of the reaction chamber of the sampling unit, fluorescence captured by the optics, or the like. In some embodiments, the processor 210 can receive data from the manipulation module 206 indicating which manipulations have been completed and/or of the status of one or multiple manipulations being completed. In some embodiments, the processor 210 can receive data from the sampling module 208, which data can indicate, for example, a flow rate, a volume of fluid passed through a sampling unit, or the like. In some embodiments, the processor 210 can generate signals controlling, for example, the analysis module 204 to perform a nucleic acid amplification process and/or to identify target analyte in the sample. In some embodiments, the processor 210 can generate signals controlling, for example, the manipulation module 206 to place a sampling unit in the sampling module 208, remove a sampling unit from the sampling module 208 after completion of the sample, place a sampling unit in the analysis module, manipulate one or multiple features of the sampling unit to perform the analysis, removing the sampling unit from the analysis module 206 after completion of the analysis, and disposing of the sampling unit. In some embodiments, the processor 210 can generate signals controlling, for example, the flow rate of the sampling module 208.

The processor 210, may be implemented as one or more integrated circuits (e.g., a conventional microprocessor or microcontroller). One or more processors, including single core and/or multicore processors, may be included in the processor 210. Processor 210 may be implemented as one or more independent processing units with single or multicore processors and processor caches included in each processing unit. In other embodiments, processor 210 may also be implemented as a quad-core processing unit or larger multicore designs (e.g., hexa-core processors, octo-core processors, ten-core processors, or greater).

Processor 210 may execute a variety of software processes embodied in program code, and may maintain multiple concurrently executing programs or processes. At any given time, some or all of the program code to be executed can be resident in processor(s) 210 and/or in memory 212. In some embodiments, sampling and analysis system 102 can include one or more specialized processors, such as digital signal processors (DSPs), outboard processors, graphics processors, application-specific processors, and/or the like.

The sampling and analysis system 102 can comprise memory 212, comprising hardware and software components used for storing data and program instructions, such as system memory and computer-readable storage media. The system memory and/or computer-readable storage media may store program instructions that are loadable and executable on processor 210, as well as data generated during the execution of these programs.

System memory may be stored in volatile memory (such as random access memory (RAM)) and/or in non-volatile storage drives (such as read-only memory (ROM), flash memory, etc.). The RAM may contain data and/or program modules that are immediately accessible to and/or presently being operated and executed by processor 210. In some implementations, system memory may include multiple different types of memory, such as static random access memory (SRAM) or dynamic random access memory (DRAM). In some implementations, a basic input/output system (BIOS), containing the basic routines that help to transfer information between elements within the sampling and analysis module 102, such as during start-up, may typically be stored in the non-volatile storage drives. By way of example, and not limitation, system memory may include application programs, such as client applications, Web browsers, mid-tier applications, server applications, etc., program data, and an operating system.

Memory 212 also may provide one or more tangible computer-readable storage media for storing the basic programming and data constructs that provide the functionality of some embodiments. Software (programs, code modules, instructions) that when executed by a processor provide the functionality described herein may be stored in memory 212. These software modules or instructions may be executed by processor 210. Memory 212 may also provide a repository for storing data used in accordance with the present invention.

Memory 212 may also include a computer-readable storage media reader that can further be connected to computer-readable storage media. Together and, optionally, in combination with system memory, computer-readable storage media may comprehensively represent remote, local, fixed, and/or removable storage devices plus storage media for temporarily and/or more permanently containing, storing, transmitting, and retrieving computer-readable information.

Computer-readable storage media containing program code, or portions of program code, may include any appropriate media known or used in the art, including storage media and communication media, such as, but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computers readable media, such as data signals, data transmissions, or any other medium which can be used to transmit the desired information and which can be accessed by processor 210.

By way of example, computer-readable storage media may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media may include, but is not limited to, Zip® drives, flash memory cards, universal serial bus (USB) flash drives, secure digital (SD) cards, DVD disks, digital video tape, and the like. Computer-readable storage media may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for the sampling and analysis system 102.

The sampling and analysis system 102 can include a communications module 214. The communications module can be configured to send information to and/or receive information from other systems including wired or wireless transmission, using standard, commercially available components such as receivers, transceivers, transmitters, modems, ethemet ports, wifi routers or the like. In some embodiments, the communications module can be configured to electronically transmit information to another system and optionally for receiving information such as operating instructions.

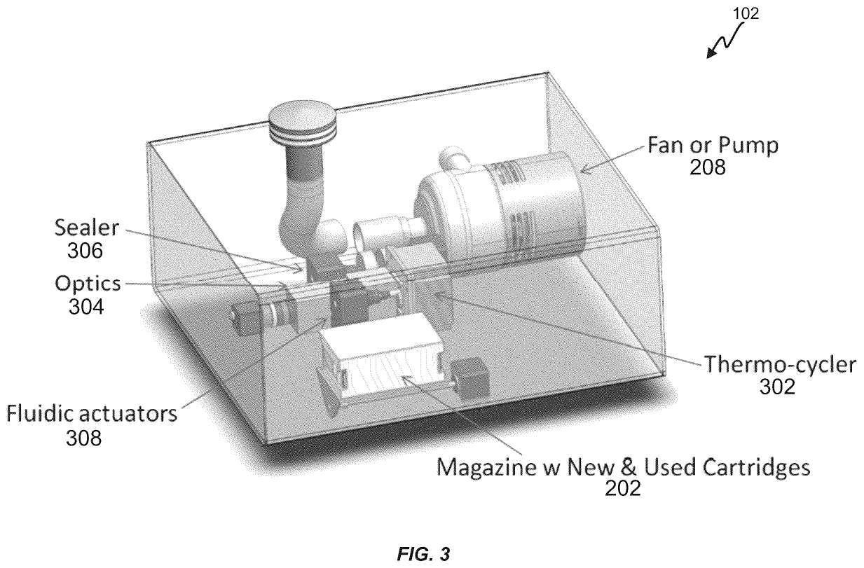

With reference now to FIG. 3, a depiction of one embodiment of the sampling andanalysis system 102 is shown. As seen the sampling and analysis system 102 includes a magazine 202 which can be configured to hold one or multiple sampling units. In some embodiments, the magazine 202 and be configured to hold one or multiple new and/or one or multiple spent sampling units. In some embodiments, for example, after the completion of analysis with a sampling unit, the spent sampling unit can be disposed of in a portion of the magazine 202 designated for spent sampling units. Nucleic acid remaining in spent sampling units can be further analyzed to obtain additional data about the environment from which the sample was obtained.

The sampling and analysis system 102 can include a thermocycler 302. The thermocycler 302 can be configured to control the temperature of the sampling unit during analysis. This can include heating and/or cooling the sampling unit, and in some embodiments, cyclically heating and/or cooling the sampling unit. In some embodiments, the thermocycler 302 can be configured to maintain a desired temperature of the sampling unit for a desired period of time. In some embodiments, for example, after a sampling unit has emplaced within the analysis module 204, the thermocycler 302 can heat and/or call the sampling unit according to one or multiple temperature profiles.

In some embodiments, the heating and/or cooling of the sampling unit can include the heating and/or cooling of a reaction chamber defined by the sampling unit. The heating and/or cooling of the reaction chamber can be cyclical and/or can be part of a nucleic acid amplification process. In some embodiments, the heating and/or cooling of the reaction chamber can include the heating and/or cooling of the SCS which can be contained within the reaction chamber. In some embodiments, with the SCS positioned in the reaction chamber, the nucleic acid amplification process can be performed directly on the SCS. Performing a nucleic acid amplification process directly on the SCS achieves higher sensitivity than rinsing or extracting analyte off of the SCS and transferring it into the reaction chamber, which would result in analyte yield losses. This allows for shorter sampling times before analysis and detection of very low amounts of analyte from the fluid. Preferred materials for SCS exhibit limited or no detectable autofluorescence.

The thermocycling of the sampling unit can include, for example, holding the reaction chamber of the sampling unit to a temperature between 20° C. and 60° C. during a reverse transcription step that is prior to a PCR amplification phase. In some embodiments, a target nucleic acid of the target analyte becomes available for exposure to reagents during sample capture and reverse transcription steps. After completion of the reverse transcription phase, the thermocycler 302 can be configured to cyclically heat and/or cool the reaction chamber of the sampling unit at temperatures between 4° C. and 100° C. Many analytes such as respiratory viruses can be detected without any extraction or other enrichment step after being captured on an SCS. Some analytes that contain encapsulated nucleic acid that is not released by a 20° C. to 60° C. step can be analyzed by, for example, holding the reaction chamber at 95° C. to 100° C. for a length of time such as 30 seconds to 5 minutes or longer before thermocycling begins. Use of detergents can also aid in analysis without requiring a separate extraction or other enrichment step.

The sampling and analysis system 102 can include optics 304. The optics 304 can be configured to send and receive electromagnetic radiation into and emitted from the reaction chamber and/or the contents of the reaction chamber, respectively, to thereby identify the presence, absence, and/or concentration of one or multiple target analytes within the reaction chamber of the sampling unit. In other words, the optics can be configured to send to and receive from and thereby measure florescent signal from the sampling reaction chamber, which reaction chamber includes the SCS. Thus, in some embodiments, the optics can be configured to send and receive and/or measure fluorescent signal to and from the reaction chamber, respectively, including any fluorescent signature received from the SCS contained in the reaction chamber.

In some embodiments, the optics can comprise one or multiple electromagnetic radiation emitters, one or multiple electromagnetic radiation detectors, or the like. In some embodiments, the one or multiple electromagnetic radiation emitters can be configured to emit excitation energy to excite fluorescence of one or multiple probes coupled to target analyte. In some embodiments, the one or multiple electromagnetic radiation detectors can be configured to receive and/or detect electromagnetic radiation emitted by one or multiple probes coupled to target analyte in the reaction chamber of the sampling unit.

In some embodiments, the optics 304 can be configured to measure at least four fluorescent channels of distinct fluorescent wavelength. In some embodiments, each of the fluorescent channels can be configured to measure a fluorescent signal, and thus optics 304 with four fluorescent channels can be configured to measure four distinct fluorescent signals.

In some embodiments, the sampling and analysis system 102 can include a sealer 306. The sealer 306 can be configured to seal one or more fluidic channels of the sampling unit and/or to seal one or more sealing members of the cartridge receiver to the cartridge body to thereby enclose the reaction chamber of the cartridge body around the SCS. In some embodiments, the sealer 306 can be configured to seal the sealing members to the cartridge body around the perimeter of the reaction chamber to thereby enclose the reaction chamber around the SCS. In some embodiments, the sealer 306 can be configured to seal the one or more fluidic channels of the sampling unit and/or one or more of the sealing members of the cartridge receiver to the cartridge body to thereby enclose the reaction chamber of the cartridge body via, for example, at least one of: heat, vibration, pressure, or the like. For example, heat can be applied to activate an adhesive that provides a stable, watertight seal once it cools.

In some embodiments, the sampling and analysis system 102 can include one or multiple fluidic actuators 308. The one or multiple fluidic actuators 308 can be configured to control the release, mixing, and movement of one or multiple reagents within one or multiple fluidic channels and chambers of the cartridge body. In some embodiments, the one or multiple reagents can be contained within one or multiple containment capsules on the cartridge body. These one or multiple containment capsules can each be configured to hold one or multiple reagents. The one or multiple containment capsules can include a first containment capsule containing one or multiple wet reagents, also referred to herein as aqueous reagents, and a second containment capsule containing one or multiple dry reagents. In some embodiments, the one or multiple fluidic actuators 308 can combine the aqueous reagents with the dry reagents, mix them to become a single homogeneous solution, and can fill the reaction chamber with the mixed reagents.

In some embodiments, the sampling and analysis system 102 can include the sampling module 208 which can include a fan, pump, vacuum, compressor, or the like. The sampling module 208 can be configured to draw fluid for sampling through the sampling unit to thereby capture sample on a filter membrane, also referred to herein as a sample capture substrate (SCS). In some embodiments, the sampling module 208 can be configured to achieve a pressure drop across the filter membrane of between approximately 1 kPa and approximately 30 kPa.

In some embodiments, and as previously discussed, the processor 210 can be configured to receive inputs from the modules and/or components of the sampling and analysis system 102 and can generate one or multiple control signals based on those received inputs and based on instructions stored in the memory 212. In some embodiments, these instructions can direct the modules and/or components of the sampling and analysis system 102 to place a new sampling unit in the fluid transfer module 208 such that the fan, pump, vacuum, compressor, or the like in the sampling module 208 can move fluid such as air through the sampling unit, and specifically through the SCS of the sampling unit to thereby collect a sample. These instructions can, for example, cause the manipulation module 206 to move the sampling unit from the fluid transport module 208 to the analysis module 204 and for the sealer 306 to enclose the reaction chamber of the sampling unit by sealing the sealing members of the cartridge receiver to the cartridge body. These instructions can further cause the manipulation module 206 to combine and mix reagents and fill the reaction chamber with the mixed reagents. These instructions can further direct the analysis module 204 to perform the nucleic acid amplification process and to determine the presence, absence, and/or concentration of target analyte in the reaction chamber. Specifically, these instructions can direct the thermocycler 302 to heat and/or cool the sampling unit, and specifically to heat the reaction chamber the sampling unit according to a desired nucleic acid amplification program These instructions can further direct the optics 304 to emit electromagnetic radiation into the reaction chamber and gather electromagnetic radiation from the reaction chamber, whereby the presence, absence, and/or concentration of target analyte in the reaction chamber can be determined.

In some embodiments, for example, the processor can be configured to control the sampling module 208 to move air through the sampling unit, and specifically through the SCS and the reaction chamber at a desired flow rate. In some embodiments, the flow rate can be, for example, between 0.1 LPM/mm² and 10 LPM/mm² through the SCS. In some embodiments, the flow rate can be between 10 LPM and 500 LPM, and/or can be approximately 100 LPM. In some embodiments, at flow rates between 0.1 LPM/mm² and 10 LPM/mm² through the SCS, the sampling and analysis system 102 can be configured to generate less than 50 Db at a distance of 36 inches from the system 102.

In some embodiments, the sampling and analysis system 102 can be configured to collect between 1 and 100 samples per day, between 10 and 80 samples per day, between 20 and 50 samples per day, approximately 32 sample per day, or any other or intermediate value. In some embodiments, at least some of these samples can be collected by moving between approximately 500 L of air through the SCS and the reaction chamber and 100,000 L of air through the SCS and the reaction chamber. In some embodiments, at least some of these samples can be collected by moving approximately or at least 3,000 L of fluid through the SCS of the sampling unit.

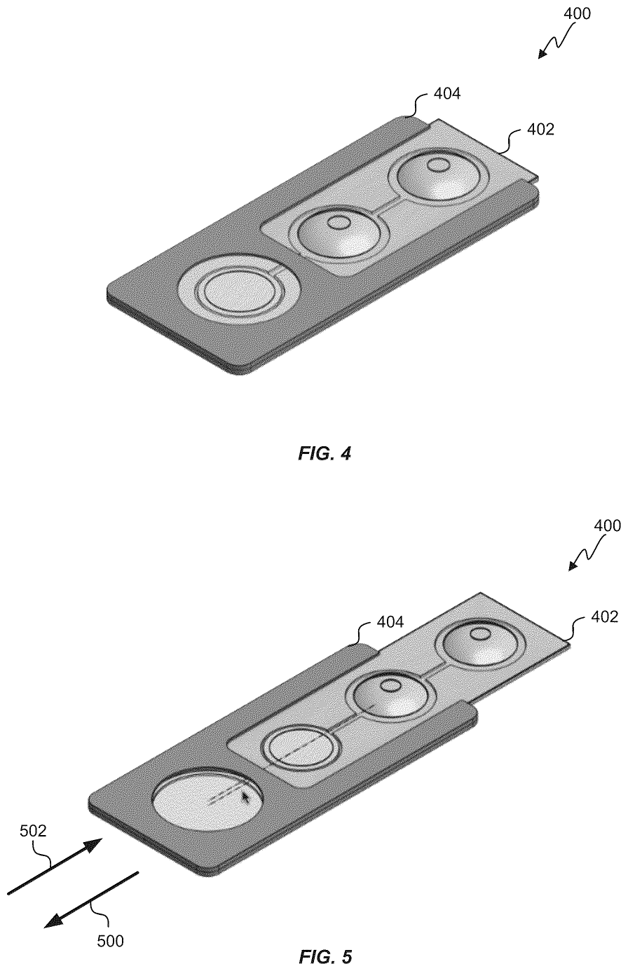

With reference now to FIG. 4, a perspective view of one embodiment of the sampling unit 400 is shown. The sampling unit 400 can, in some embodiments, be configured to receive and/or collect a sample. In some embodiments, the sampling unit 400 can be configured to collect a sample from a fluid, such as from the air, or from a liquid. The sampling unit 400 can be, for example, disposable or can be reusable.

The sampling unit 400 can comprise a consumable body comprising a cartridge body 402, and a cartridge receiver 404. One of the cartridge body 402 and the cartridge receiver 404 can be referred to as a first portion of the sampling unit 400 and the other of the cartridge body 402 and the cartridge receiver 404 can be referred to as a second portion. In some embodiments, the cartridge body 402 and the cartridge receiver 404 can be coupled, such as via a hinge, a pivot, a slide, or the like, and in some embodiments, the cartridge body 402 and the cartridge receiver 404 can be separate, or in other words, are not connected.

In some embodiments, and as depicted in FIG. 5, the cartridge body 402 can be wholly or partially separated from the cartridge receiver 404. In some embodiments, the cartridge body 402 can be inserted into the cartridge receiver 404, and specifically into a receptacle defined by the cartridge receiver 404 by moving the cartridge body 402 with respect to the cartridge receiver 404 as indicated by arrow 500. In some embodiments, the cartridge body 402 can be removed from the cartridge receiver 404, and specifically can be removed from a receptacle defined by the cartridge body 404 by moving the cartridge body 402 with respect to the cartridge receiver 404 as indicated by arrow 502. The ability to move the cartridge body 402 with respect to the cartridge receiver 404 allows the sampling unit 400 to collect analyte on the SCS while in a first position, wherein the fan or pump 208 flows fluid such as air through the SCS. Once the sample has been captured, the manipulation module 206 moves the cartridge body 402 into a second position wherein the SCS is no longer in position to have fluid flowing through the SCS, but instead places the SCS into position to have sealing members seal around the reaction chamber and SCS, such that nucleic acid amplification reagents can be moved into the reaction chamber by the fluid transport module 208 and nucleic acid amplification can be performed directly on the SCS.

The cartridge body 402 can be moved by the manipulation module 206 to the first position, to the second position, to a position to collect a sample, to a position for nucleic acid amplification and/or analysis, and/or into and/or out of the magazine 202. In some embodiments, this can include rotating the cartridge body 402 about one or several axes; longitudinally displacing the cartridge body 402 along one or several axes, or the like. In some embodiments, for example, the cartridge body 402 can be placed in the first position and can be inserted into a gap in channel for fluid flow such that the fluid flowing through the channel passes through the SCS. In some embodiments, this insertion can include a rotation, a longitudinal displacement, and/or the like.

After the sample has been collected, the manipulation module 206 can remove the cartridge body 402 from the channel for fluid flow and place the cartridge body 402 in the second position. In some embodiments, the manipulation module 206 can position the cartridge body 402 such that the sealer 306 can seal the sealing members 632 to the cartridge body 402 to thereby enclose the reaction chamber 606. The manipulation module 206 can position the cartridge body 402 with respect to the analysis module 204 such that the nucleic acid amplification and analysis can be performed. In some embodiments, this can include inserting the cartridge body 402 into a position to contact the thermocycler 302 and/or the optics 304. In some embodiments, the cartridge body 402 can be longitudinally displaced and/or rotated to come to this position. In some embodiments, one or both of the thermocycler 306 and the optics 304 can move with respect to the cartridge body 402. After the nucleic acid amplification and/or analysis has been completed, the manipulation module 206 can place the used sampling unit 400 in the magazine 202, and specifically in the portion of the magazine for containing used sampling units 400.

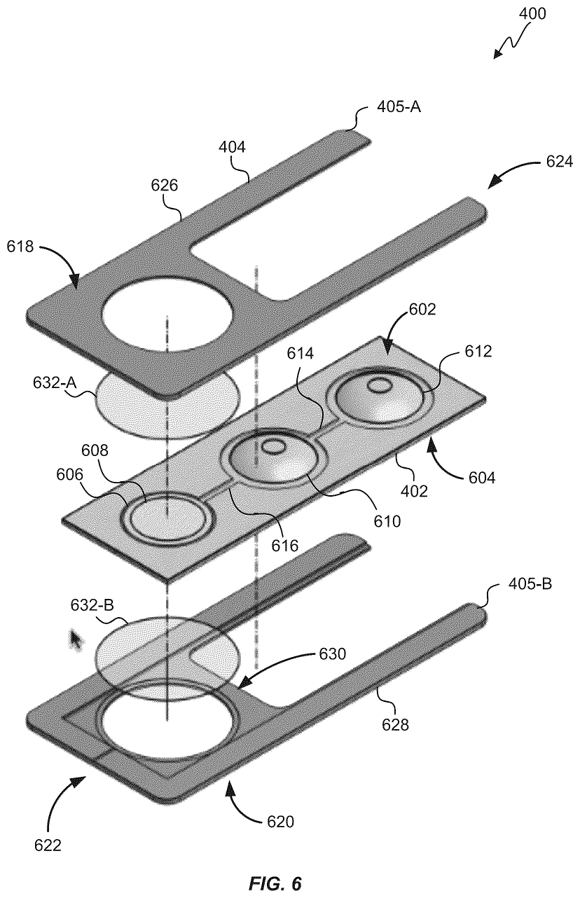

With reference now to FIG. 6, an exploded view of one embodiment of the sampling unit 400 is shown. The cartridge body 402 can comprise a variety of shapes and sizes and can be made from a variety materials. In some embodiments, the cartridge body 402 can comprise metal, a man-made material, or natural material. In some embodiments, the cartridge body can comprise, for example, a man-made material such as a polymer such as polypropylene, polyethylene, polycarbonate, polytetrafluoroethylene (PTFE), or the like.

As seen, the cartridge body 402 can comprise a front 602 and a back 604. While the present application describes and/or depicts certain features as being on one of the front 602 and the back 604, some or all of these features can, in some embodiments, be located on the other of the front 602 and the back 604. In some embodiments, the front 602 and the back 604 of the cartridge body 402 can include the same features and/or can be identical. In such an embodiment, the orientation of the cartridge body 402 is unimportant. In some embodiments, the front 602 can be different from the back 604 of the cartridge body 402. In some embodiments, the cartridge body 402 can be configured to collect a sample via flow of fluid such as air through a reaction chamber 606, also referred to herein as an air sampling reaction chamber, defined by the cartridge body 402 wherein the air flows from the front 602 of the cartridge body 402 to the back 604 of the cartridge body 402. The reaction chamber 606 can comprise an enclosable chamber. In some embodiments, the reaction chamber 606 can be integrated into the cartridge body 402.

The reaction chamber 606 can comprise a variety of shapes and sizes. The reaction chamber 606 can be defined by the cartridge body 402. In some embodiments, for example, the reaction chamber 606 extends through the cartridge body 402, and specifically extends through the front 602 of the cartridge body 402 and/or through the back 604 of the cartridge body 402. The reaction chamber 606 can comprise a variety of shapes and sizes and can be, for example, circular, rectangular, triangular, or have any other desired shape.

In some embodiments, the reaction chamber 606 can have a width, a side length, and/or a diameter of between approximately 1 mm and approximately 50 mm. In some embodiments, for example, the reaction chamber 606 can be circular and can have a diameter of between approximately 1 mm and approximately 50 mm. In some embodiments, the reaction chamber 606 can have a width, a side length, and/or a diameter of approximately 12 14 mm. The reaction chamber 606 can have a width, a side length, and/or a diameter larger than 50 mm, such as 500 mm or more, however a larger the reaction chamber requires more nucleic acid amplification reagents and a larger thermocycling module 302.

A filter membrane 608, also referred to herein as a “Sample Capture Substrate (SCS) 608” or as “SCS 608”, can be connected and/or coupled to the cartridge body 402. The SCS 608 can be configured to collect, contain, and/or hold a sample. In some embodiments, the SCS 608 can be coupled to the cartridge body 402, and in some embodiments, the SCS 608 can be separate from the cartridge body 402. The SCS 608 can comprise a variety of shapes and sizes and can be made from a variety of materials. In some embodiments, the SCS 608 comprises at least one of: a membrane; a swab; a brush; and a scoop.

In some embodiments, the sample capture substrate 608 can be coupled to the cartridge body 402 around a perimeter of the air sampling reaction chamber 606. The SCS 608 can extend across the reaction chamber 606. In some embodiments, the filter membrane 608 extends across the reaction chamber 606 such that the reaction chamber 606 is completely covered by the filter membrane 608 such that there are no fluid paths through the reaction chamber 606 except through the filter membrane 608.

In some embodiments, the filter membrane 608 can have a width, a side length, or diameter equal to the width or diameter of the reaction chamber 606. Thus, in some embodiments, the filter membrane 608 can have a width, a side length, and/or a diameter of approximately 1 mm and approximately 50 mm. In some embodiments, the filter membrane 608 can have a width, a side length, and/or a diameter of approximately 12 mm. In some embodiments, the filter membrane 608 can have an area of between approximately 0.5 mm² and approximately 2,000 mm², and in some embodiments, the filter membrane 608 can have an area of approximately 115 mm². In some embodiments, the filter membrane 608 can have at least one of: an area of approximately 115 mm²; and a width or diameter of approximately 12 mm. The filer membrane 608 can have an area larger than 2,000 mm², such as 20,000 mm² or larger, however a larger reaction chamber requires more nucleic acid amplification reagents, a larger thermocycling module 302, and will be less sensitive.

The filter membrane 608 can be configured to collect a sample from the fluid passed through the filter membrane 608 and through the reaction chamber 606. This can include, for example, capturing biological matter including biological pathogens such as allergens, mold, bacteria, viruses, or the like from fluid such as air passed through the filter membrane 608. In some embodiments, the filter membrane can be configured to capture airborne microbes from air passed through the filter membrane 608.

The filter membrane 608 can comprise a variety of materials. In some embodiments, the filter membrane 608 can comprise a material configured to capture sample passed through the reaction chamber 606. In some embodiments, the filter membrane 608 can comprise at least one of polytetrafluoroethylene (PTFE), polycarbonate, and a propylene such as, for example, polypropylene. In some embodiments, the filter membrane 608 can comprise melt-blown polypropylene. In some embodiments, the filter membrane 608 can be electrostatically charged, and in some embodiments, the filter membrane 608 is not electrostatically charged.

The filter membrane 608 can comprise a variety of thicknesses and/or can comprise a variety of different material weights. In some embodiments, the filter membrane 608 can have a weight of, for example, between approximately 1 g/m² and 100 g/m², and in some embodiments, the filter membrane can have a weight of, for example, between approximately 5 g/m² and approximately 60 g/m². In some embodiments, the filter membrane 608 can have a thickness of between approximately 0.02 mm and 0.5 mm. The filter membrane 608 can have a thickness of greater than 0.5 mm, such as 3 mm or thicker, however increasing thickness increases pressure drop and therefore noise and requires the air conveying tubing leading to the reaction chamber 606 to be stronger. Increasing thickness also requires a larger volume reaction chamber, which slows down the thermocycling process.

In some embodiments, the filter membrane 608 can be selected for compatibility with the sampling and analysis system 102. In some embodiments, this can include compatibility with the nucleic acid amplification process performed by the sampling and analysis system 102. Compatibility with the nucleic acid amplification process can include, for example a material that is compatible with temperatures of the nucleic acid amplification process, a material that is compatible with chemicals and/or reagents used in the nucleic acid amplification process, a material that does not interfere with the nucleic acid amplification process, and/or a material that does not interfere with detection of target analytes in the sample. In some embodiments, a preferred filter membrane 608 material meets some or all of the aforementioned requirements.

In some embodiments, the filter membrane 608 can be compatible with the temperatures the nucleic acid amplification process, and specifically can be stable at temperatures between approximately 4° C. and approximately 100° C. In some embodiments, the filter membrane 608 can be compatible with the nucleic acid amplification process in that the filter membrane 608 does not inhibit nucleic acid amplification reactions such as, for example, PCR reactions.

In some embodiments, the filter membrane 608 can be compatible with the nucleic acid amplification process in that the filter membrane 608 does not inhibit detection of target analytes within the reaction chamber 606. In some embodiments, the filter membrane 608 does not inhibit detection of target analytes within the reaction chamber 606 due to the autofluorescence of the filter membrane 608. For example, in some embodiments, the filter membrane 608 exhibits no autofluorescence, or autofluorescence at a level that does not interfere with quantitative nucleic acid amplification analysis, and specifically does not interfere with quantitative nucleic acid amplification analysis that uses florescent probes for target identification. In some embodiments, the filter membrane does not interfere with quantitative nucleic acid amplification analysis when the filter membrane generates less than, for example, 5%, 10%, 15%, 20%, 25%, or 30 of total system fluorescence. In some embodiments, the filter membrane does not interfere with quantitative nucleic acid amplification analysis when the sample capture substrate exhibits fluorescence ofless than 50% of the baseline fluorescence. In some embodiments, the filter membrane does not interfere with quantitative nucleic acid amplification analysis because the filter membrane demonstrates no autofluorescence.

The cartridge body 402 can include one or multiple containment capsules that can contain one or multiple reagents for use in the nucleic acid amplification process. In some embodiments, the cartridge body 402 can include a first containment capsule 610 that can contain first reagents and the second containment capsule 612 that can contain second reagents. In some embodiments, the first reagents contain in the first containment capsule 610 can comprise one or multiple dry reagents. In some embodiments, the second reagents contained in the second containment capsule 612 can comprise one or multiple aqueous reagents, also referred to herein as “wet reagents.” In some embodiments, the dry reagents can comprise, for example, lyophilized nucleic acid amplification reagents such as, for example, lyophilized nucleic acid amplification reagents (primers, fluorescent probes, reverse transcriptase, dNTPs, DNA polymerase, and various buffer components), and in some embodiments, the aqueous reagents can comprise aqueous nucleic acid amplification reagents, and specifically can include aqueous PCR reagents. In some embodiments, at least one of the reagents comprises a surfactant. The surfactant can, in some embodiments comprise a nonionic surfactant, an ionic surfactant, or a combination of one or multiple nonionic surfactants and/or one or multiple ionic surfactants. In some embodiments, the surfactant can comprise at least one of: Tween; NP40; and Triton X-100. Non-specific nucleic acid amplification can be prevented by mixing all necessary reagents at a temperature at or above the annealing and extension temperature of the PCR reaction, thus sequestering and/or separating critical reagents such as (but not limited to) Mg²+ from polymerases until the reaction is at the required temperature or until the reagents are ready to be transferred to the reaction chamber. Another approach is to block the activity of reagents such as (but not limited to) the enzyme. This can also be achieved by using reversible, temperature-dependent inhibitors such as antibodies or aptamers that are inactivated at or above the annealing and extension temperature.

The nucleic acid amplification reagents chosen for inclusion in a cartridge determine which biological entities such as microbes can be identified by using that cartridge in the sampling and analysis system 102. For example, PCR primers specific for certain respiratory viruses and bacteria can be used in systems placed in indoor environments such as office buildings, public transportation hubs such as airports, and other built environments containing humans including hospitals, ships, and residences. Other microbes than can be analyzed include fungi such as toxic and allergenic molds that can accumulate in buildings and microbes that can cause hospital acquired infections. Non-microbial sources of nucleic acid can also be analyzed using certain primers, such as primers used to identify certain plant species that produce allergenic pollen. Primers can also be used to identify specific humans in the vicinity of the sampling and analysis system 102, such as primers that recognize the 13 core short tandem repeat loci that are used in the identification of individuals in the United States by law enforcement agencies such as the FBI using the CODIS (Combined DNA Index System) by amplifying human cells entrained in the air exiting the respiratory tract. Target organisms and viruses can include one or more of Clostridium botulinum, Listeria, Campylobacter, Trichinosis, Staphylococcus aureus including methicillin and vancomycin-resistant Staphylococcus aureus, Pseudomonas aeruginosa, Klebsiella pneumonia, Mycobacterium tuberculosis, Neisseria gonorrhoeae, Enterococcus including vancomycin-resistant Enterococcus, Salmonella including fluoroquinolone-resistant Salmonella, C. difficile including clindamycin-resistant C. difficile, Bacillus anthracis, A baumannii including multidrug-resistant A baumannii, Streptococcus pneumonia, Candida albicans, Pseudomonas aeruginosa, Acinetobacter baumannii, Stenotrophomonas maltophilia, E. coli including fluoroquinolone-resistant E. coli and E. coli O157:H7, Legionella pneumophila, Streptococcus pyogenes, Ebola virus, dengue virus, novavirus, viruses that cause Lassa fever, yellow fever, Marburg hemorrhagic fever and Crimean-Congo hemorrhagic fever, rhinovirus including types A, B and C, coronavirus including SARS CoV, SARS CoV-2 (including Alpha (B.1.1.7 and Q lineages), Beta (B.1.351 and descendent lineages), Gamma (P. 1 and descendent lineages), Epsilon (B.1.427 and B.1.429), Eta (B.1.525), Iota (B.1.526), Kappa (B.1.617.1 and 1.617.3), Mu (B.1.621, B.1.621.1), Zeta (P.2), Delta (B.1.617.2 and AY lineages) and Omicron (B.1.1.529, BAI, BAI.1 and BA2 lineages)) and MERS, adenovirus, influenza virus including types A and B; parainfluenza virus; respiratory syncytial virus (including types A and B); enterovirus; norovirus (including genogroups GI, Gli, GIii, GIV, GV, GVI, and GVII); rotavirus, astrovirus, viruses that cause measles, rubella, chickenpox/shingles, roseola, smallpox and fifth disease, chikungunya virus, hepatitis (including types A, B, C, D, and E), herpesvirus, papilloma virus; molluscum contagionsum; polio virus; rabies virus, viruses that cause viral meningitis and encephalitis, HIN1, HIN2, H2N2, H2N3, H3NI, H3N2, H3N8, H5NI, H5N2, H5N3, H5N6, H5N8, H5N9, H6NI, H6N2, H7NI, H7N2, H7N3, H7N4, H7N7, H7N9, H9N2, HION7, HI0N8, HIIN2, HIIN9, Hl7NI0, Hl8NII, HPIV-1, HPIV-2, HPIV-3, HPIV-4, HAdV-B, HAdV-C, 229E, OC43, NL63, HUKI, Sin Nombre orthohantavirus, Black Creek Canal orthohantavirus, Puumala virus, Thaland virus; HRV-AI, HRV-A2, HRV-A7-13, HRV-AJ5, HRV-A16, HRV-A18-25, HRV-A28-34, HRV-A36, HRV-A38-41, HRV-A43-47, HRV-A49-51, HRV-A53-68, HRV-A71, HRV-A73-78, HRV-A80-82, HRV-A85, HRV-A88-90, HRV-A94-96, HRV-A98, HRV-AJ00-103, HRV-B3-6, HRV-B14, HRV-B17, HRV-B26, HRV-B27, HRV-B35, HRV-B37, HRV-B42, HRV-B48, HRV-B52, HRV-B69, HRV-B70, HRV-B72, HRV-B79, HRV-B83, HRV-B84, HRV-B86, HRV-B91-93, HRV-B97, HRV-B99, and HRV-CJ-51. Primers can also be used that are specific for human associated microbes such as Actinomyces, Aerococcus, Akkermansia, Alistipes, Alloiococcus, Anaerococcus, Anaerotruncus, Atopobium, Bacteroides, Barnesiella, Bifidobacterium, Blautia, Butyrivibrio, Chlamydia, Clostridium, Corynebacterium, Cutibacterium (formerly Propionibacterium), Dialister, Dysgonomonas, Enterobacter, Enterococcus, Escherichia, Faecalibacterium, Fusobacterium, Gardnerella, Gemella, Haemophilus, Klebsiella, Kocuria, Lactobacillus, Lactococcus, Megasphera, Methanobrevibacter, Micrococcus, Mobiluncus, Moraxella, Mycobacterium, Mycoplasma, Neisseria, Oxalobacter, Papillibacter, Parabacteriodes, Parvimonas, Peptoniphilus, Peptostreptococcus, Porphyromonas, Prevotella, Pseudomonas, Roseburia, Ruminococcus, Sneathia, Spirochaeta, Staphylococcus, Streptococcus, Villonella, Alternaria, Aspergillus, Candida, Cladosporium, Curvularia, Embellisia, Fusarium, Penicillium, Saccharomyces, Stachybotrys, Thermomyces, Trichophyton, Malassezia, and Rhodotorula. Primers can also be used that are specific for human RNAse-P. Primers can also be used that are specific for allergenic molds including Alternaria, Aspergillus (including A. Jumigatus, A. versicolor and A. jlavus), Cladosporium, Cryptococcus (including Cryptococcus neoformans), Histoplasma capsulatum, Stachybotrys (including Stachybotrys chartarum), Penicillium (including P. brevicompactum, P. chrysogenum, P. citrinum, P. corylophilum, P. cyclopium, P. expansum. P. fellutanum, P. spinulosum, and P. viridicatum), Helminthosporum, Epicoccum, Fusarium (including F. solani, F. oxysporum, F. moniliforme), Aureobasidium, Phoma, Smuts, Rhizopus and Mucor. Primers can also be used that are specific for allergenic pollen including from plants and trees including ragweed, mountain cedar, ryegrass, maple, elm, mulberry, pecan, oak, pigweed/tumbleweed, Arizona cypress, alder, ash, beech, birch, box elder, cedar, cottonwood, date palm, elm, mulberry, hickory,juniper, oak, pecan, Phoenix palm, red maple, silver maple, sycamore, walnut, willow Bermuda grass, Johnson grass, Kentucky bluegrass, orchard grass, rye grass, sweet vernal grass, Timothy grass, English plantain, lamb's quarters, redroot pigweed, sagebrush and tumbleweed (Russian thistle). Those skilled in the art also recognize that universal primers capable of recognizing all organisms at a certain taxonomic level such as phylum, class, order, family, genus, species and strain. For example, fungi (see J. Nat. Prod. 2017, 80, 3, 756-770) and coronaviruses (J Clin Microbiol. 2007 March; 45(3): 1049-1052).

In some embodiments, the first containment capsule 610 and the second containment capsule 612 are connected by a first fluidic channel 614 in the cartridge body 402. In some embodiments, the first containment capsule 610 is further connected with the reaction chamber 606 and the filter membrane 608 via a second fluidic channel 616 in the cartridge body 402. In some embodiments, the second containment capsule 612 can be compressed to move some or all of the wet reagents in the second containment capsule 612 through the first fluidic channel 614 to mix with the dry reagents in the first containment capsule 610. After the aqueous reagents have successfully mixed with the dry reagents, the first containment capsule 610 can be compressed to move some or all of the mixed reagents from the first capsule 610 into the reaction chamber 606 thereby wetting and/or immersing the filter membrane in the mixed reagents.

The cartridge receiver 404 can comprise a variety of shapes and sizes and can be made from a variety of materials. In some embodiments, for example, the cartridge receiver can comprise a man-made material such as a polymer including, for example, polypropylene, polycarbonate, or the like.

The cartridge receiver 404 can comprise a front 618, a back 620, a bottom 622, a top 624, a first side 626, and the second side 628. The cartridge receiver 404 can define a receptacle 630 configured to receive all or portions of the cartridge body 402, and specifically to enclose the reaction chamber 606 and/or the SCS 608. In some embodiments, the receptacle 630 can comprise an enclosable chamber. The cartridge receptacle 630 can be accessed via the top 624 of the cartridge receiver 404. The cartridge receptacle 630 can, in some embodiments, be closed on all sides with the exception of an opening through the top of the cartridge receiver 404 through which all or portions of the cartridge body 402 can be inserted into the cartridge receptacle 630. When at least portions of the cartridge body 402 are inserted into the cartridge receptacle 630, the cartridge receptacle 630 can enclose the reaction chamber 606 of the cartridge body 402 to enable performing a nucleic acid amplification process. In some embodiments, the receptacle 630 of the cartridge receiver 404 is configured to enable insertion of at least the reaction chamber 606 and the filter membrane 608 into the receptacle 630 such that nucleic acid amplification and analysis can be performed on the contents of the reaction chamber 606 including on the filter membrane 608.

The cartridge receiver 404 can comprise a single piece, or to be made from a plurality pieces. In some embodiments, and is shown in FIG. 6, the cartridge receiver 404 comprises a body 405 that can have a first body piece 405-A, a second body piece 405-B, a first sealing member 632-A, and a second sealing member 632-B. The sealing members 632 can comprise a variety of shapes and sizes and can be made from a variety of materials. In some embodiments, the first sealing member 632-A can be coupled or connected to an interior surface of the front 618 of the cartridge receiver 404 and the second sealing member 632-B can be coupled or connected to an interior surface of the back 620 of the cartridge receiver 404. In some embodiments, the sealing members 632 can be coupled to the cartridge receiver 404 such that the first sealing member 632-A is positioned intermediate between the first body piece 405-A and the cartridge body 402 when the cartridge body 402 is inserted within the cartridge receptacle 630, and such that the second sealing member 632-B is positioned intermediate between the second body piece 405-B and the cartridge body 402 when the cartridge body 402 is inserted within the cartridge receptacle 630.

In some embodiments, the sealing members 632 can be made of the same material as the cartridge receiver 404, and in some embodiments, the sealing member 632 can be made from a different material than the cartridge receiver 404. In some embodiments, one or both of the sealing member 632 can be made of a man-made material such as a polymer including, for example, polypropylene, polycarbonate, or the like. In some embodiments, one or both of the sealing members 632 can be transparent. Specifically, in some embodiments, at least one of the sealing members 632 is transparent. In some embodiments, the at least the first sealing member 632-A is transparent and the sampling unit 400 can be positioned such that the transparent first sealing member 632-A exposes the reaction chamber 606 and the filter membrane 608 to the optics 304. In such an embodiment, the sampling unit 400 can be positioned such that the first sealing member 632-A is proximate to the optics 306. In such an embodiment, the second sealing member 632-B can be non-transparent, and in some embodiments, the second sealing member 632-B can have one or multiple properties to enhance heat transfer to thereby facilitate the nucleic acid amplification process. In some embodiments, for example, the second sealing member 632-B can be a good heat conductor such as copper or aluminum, and/or can comprise a color, such as black, configured to absorb heat energy.

In some embodiments, one or both of the sealing members 632 can comprise an extruded film. In some embodiments, one or both of the sealing members 632 can have a thickness between approximately 50 μm and approximately 500 μm. In some embodiments, at least one of the sealing members 632 has a thickness of less than approximately 200 μm. In some embodiments, one or both of the sealing members 632 can comprise a material that has sufficient thickness and properties to maintain rigidity during, for example, thermocycling.

In some embodiments, the sealing members 632 can each be larger than the reaction chamber 606 and can be positioned in their respective portion of the cartridge receiver 404 such that when the cartridge body 402 is fully inserted into the receiver each of the sealing members 632 covers and encloses the portion of the reaction chamber 606. In some embodiments, for example, the first sealing member 632-A covers and encloses the portion of the reaction chamber extending through the front 602 of the cartridge body 402, and the second sealing member 632-B covers and encloses the portion of the reaction chamber 606 extending through the back 604 of the cartridge body 402.

In some embodiments, each of the sealing members 632 can be configured to sealingly couple to one of the front 602 or the back 604 of the cartridge body 402. In some embodiments, each of the sealing members 632 can comprise an adhesive configured to sealingly couple the sealing member 632 to the cartridge body 402 around the reaction chamber 606. In some embodiments, this adhesive extends at least around the perimeter of the sealing member 632. In some embodiments, this adhesive can be activated, such as by heat.

In some embodiments, the adhesive can be uniformly spread across all or portions of the sealing members 632, and in some embodiments, the adhesive can be located on one or several predetermined portions of the sealing members 632. In some embodiments, for example, the adhesive can be located on the sealing members 632 such that the adhesive engages with the cartridge body 402 around the reaction chamber 606 when the sealing members 632 are sealed to the cartridge body 402. In some embodiments, the adhesive can be located in a ring of adhesive, which ring is sized, shaped, and positioned such that the adhesive engages with the cartridge body 402 around the reaction chamber 606.

In some embodiments, the sealing members 632 can be mechanically coupled to the cartridge body 402 via one or several coupling features. In such an embodiment, the sealing member 632 can comprise a gasket, such as a rubber o-ring, that can be sized to extend around the reaction chamber 606. The sealing member 632 can further include one or several features configured to engage with the cartridge body 402 to secure the sealing member 632 to the cartridge body 402 with sufficient forces such that the gasket seals to the cartridge body 402.

Each of the sealing members 632 can be configured to sealingly couple to one of the front 602 or the back 604 of the cartridge body 402 around the reaction chamber 606, and specifically around the perimeter of the reaction chamber 606 to thereby enclose the reaction chamber 606 and the filter membrane 608. In some embodiments, for example, the first sealing member 632-A is configured to sealingly couple to the front 602 of the cartridge body 402 around the reaction chamber 606, and the second sealing member 632-B is configured to sealingly couple to the back 604 of the cartridge body 402 around the reaction chamber 606. In some embodiments, sealingly coupling the first sealing member 632-A to the front 602 of the cartridge body 402 and the second sealing member 632-B to the back 606 of the cartridge body 402 can sealingly enclose the reaction chamber 606 and the filter membrane 608. In some embodiments, the enclosed reaction chamber 606 can define the volume that can be, for example, between approximately 10 μL and approximately 250 μL, between approximately 50 μL and approximately 200 μL, and/or can be approximately 100 μL. In some embodiments, the filter membrane 608 contained within the enclosed reaction chamber 606 can fill approximately 1 μL of chamber volume per mm² of filter membrane 608.

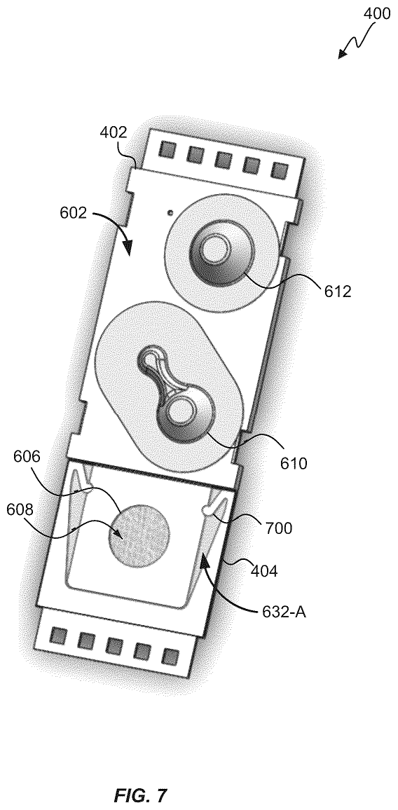

With reference now to FIG. 7, a front perspective view of another embodiment of the sampling unit 400 is shown. The sampling unit includes the cartridge body 402 defining the reaction chamber 606, which reaction chamber 606 contains the filter membrane 608. The cartridge body further includes at least one containment capsule, and specifically includes a first containment capsule 610 and a second containment capsule 612. The sampling unit 400 further includes the cartridge receiver 404. As depicted in FIG. 7, the cartridge body 402 is partially inserted within the receptacle 630 of the cartridge receiver 404 such that the reaction chamber 606 and the filter membrane 608 are enclosed within the receptacle 630 of the cartridge receiver 404. As further depicted, a sealing member 632, and specifically the first sealing member 632-A extends across the front 602 of the cartridge body 402 to enclose one side of the reaction chamber 606 and one side of the filter membrane 608.

As further seen in FIG. 7 the cartridge receiver 404 can include one or multiple locking features 700. The locking features 700 can engage with mating portions of the cartridge body 402 to secure the cartridge body 402 within the receptacle 630 of the cartridge receiver 404. In some embodiments, the locking features 700 can comprise one or multiple flexible members configured to expand into a mating feature, such as a divot located on the cartridge body 402 to couple the cartridge body 402 the cartridge receiver 404. The sampling unit 400 depicted in FIG. 7 shows the unit in an analysis configuration, whereby the reaction chamber 606 and filter membrane 608 are in position for being sealed into the reaction chamber in preparation for nucleic acid amplification.

With reference now to FIG. 8, a front perspective view of one embodiment of the sampling unit 400 is shown. The sampling unit includes the cartridge body 402 defining the reaction chamber 606, which reaction chamber 606 contains the filter membrane 608. The cartridge body further includes at least one containment capsule, and specifically includes a first containment capsule 610 and a second containment capsule 612. The sampling unit 400 further includes the cartridge receiver 404. As depicted in FIG. 7, the cartridge body 402 is partially removed from the receptacle 630 of the cartridge receiver 404 such that the reaction chamber 606 and the filter membrane 608 are not enclosed within the receptacle 630 of the cartridge receiver 404. Further, the cartridge body 402 is partially removed from the receptacle 630 of the cartridge receiver 404 such that the locking features 700 of the cartridge receiver are not engaged with the mating features 800 of the cartridge body 402. The sampling unit 400 depicted in FIG. 8 shows the unit in a sample capture configuration, whereby the reaction chamber 606 and filter membrane 608 are not inserted into the cartridge receiver 404 and are available to receive fluid flow, and specifically to receive air flow through the filter membrane 608.

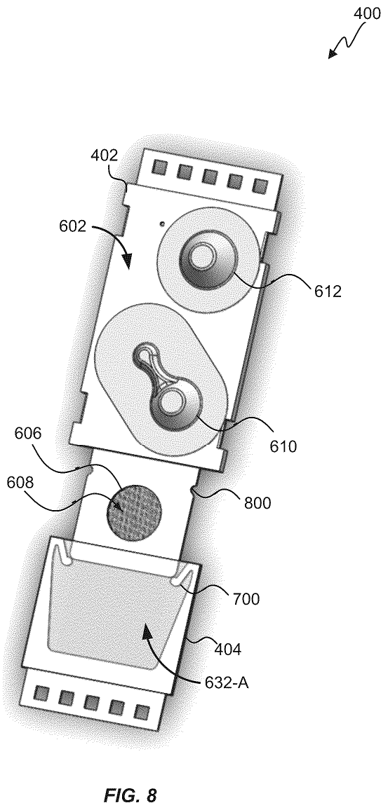

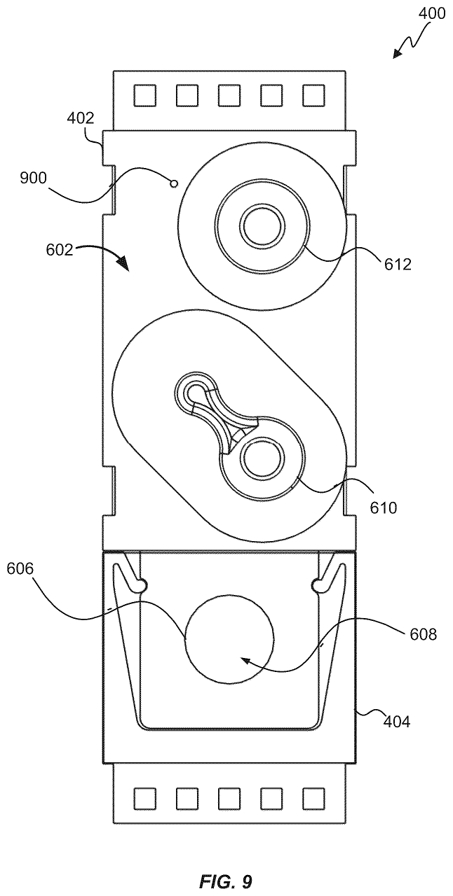

With reference now to FIG. 9, a front view of one embodiment of the sampling unit 400 is shown. The sampling unit includes the cartridge body 402 defining the reaction chamber 606, which reaction chamber 606 contains the filter membrane 608. The cartridge body further includes at least one containment capsule, and specifically includes a first containment capsule 610 and a second containment capsule 612. The sampling unit 400 further includes the cartridge receiver 404. As further depicted, the cartridge body 402 includes a bleed hole 900 configured to release pressure during the movement of reagents from the containment capsules 610, 612. In some embodiments, the bleed hole 900 can connect to a chamber and/or fluidic channel inside of the cartridge body 402, which chamber or fluidic channel inside of the cartridge body 402 is fluidly coupled to the reaction chamber and to the containment capsules 610, 612.

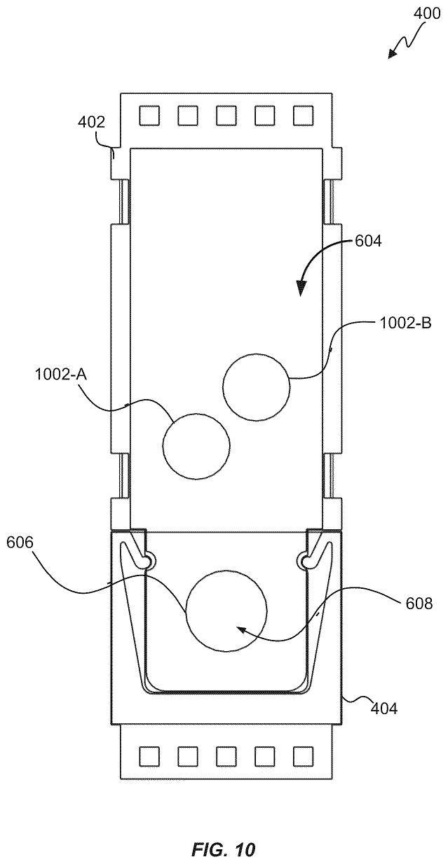

With reference now to FIG. 10, a back view of one embodiment of the sampling unit 400 is shown. The sampling unit 400 includes the cartridge body 402 defining the reaction chamber 606, which reaction chamber 606 contains the filter membrane 608. The sampling unit 400 further includes the cartridge receiver 404.

As further depicted, the back 604 of the cartridge body 402 includes one or multiple compliant puncture features 1002. In some embodiments, and as will be discussed in greater detail below, a puncture feature can release contents of a containment capsules 610, 612 to thereby allow mixing of contents of containment capsules 610, 612 and/or movement of the contents of the containment capsules 610, 612 to the reaction chamber 606. In some embodiments, the compliant puncture feature 1002 can improve the release of contents of one or multiple of the containment capsules 610, 612. In the embodiment depicted in FIG. 10, the compliant puncture feature 1002 includes a pair of compliant puncture features 1002 that are associated with the first containment capsule 610. In some embodiments, this pair of compliant puncture features 1002 includes a first compliant puncture feature 1002-A and a second compliant puncture feature 1002-B. In some embodiments, manipulation of the first compliant puncture feature 1002-A can open a portion of the first containment capsule 610 to allow mixing of the contents of the first containment capsule 610 with the contents of the second containment capsule 612. In some embodiments, manipulation of the second compliant puncture feature 1002-B can open a portion of the first containment capsule 610 to allow the contents of the first containment capsule 610, and specifically the mixed contents of the first containment capsule 610 and the second containment capsule 612 to move to the reaction chamber 606.

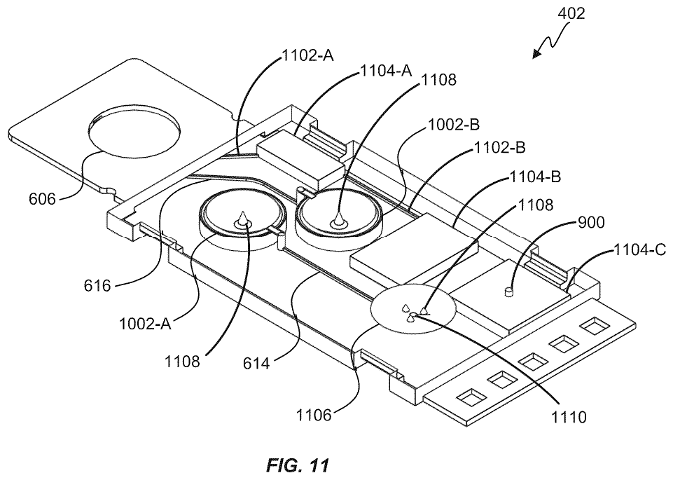

With reference now to FIG. 11, a front perspective view of a transparent cartridge body 402 is shown. The cartridge body 402 defines the reaction chamber 606, the first fluid channel 614, the second fluid channel 616, the bleed hole 900, exit channels 1102, and overflow chambers 1104. The overflow chambers 1104 can be configured to hold any excess fluid from the reaction chamber 606, and specifically can include a first overflow chamber 1104-A, a second overflow chamber 1104-B, and a third overflow chamber 1104-C. In some embodiments, and as depicted in FIG. 11, the first overflow chamber 1104-A is fluidically most proximate of all of the overflow chambers 1104, the third overflow chamber 1104-C is fluidically least proximate of all of the overflow chambers 1104, and the second overflow chamber 1104-B is intermediate between the first overflow chamber 1104-A and the third overflow chamber 1104-C to the reaction chamber 606.

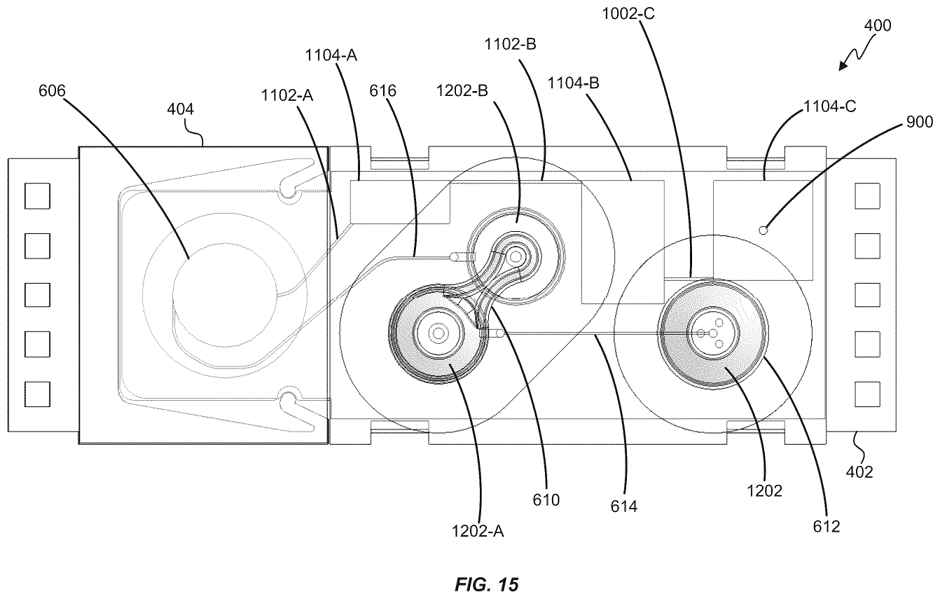

The first overflow chamber 1104-A is fluidically connected to the reaction chamber 606 via a first exit channel 1102-A. a second exit channel 1102-B fluidically connects the first overflow chamber 1104-A to the second overflow chamber 1104-B, and a third exit channel 1102-C(shown in FIG. 15) fluidically connects the second overflow chamber 1104-B to the third overflow chamber 1104-C. Further, the third overflow chamber 1104-C is connected to the external environment via the bleed hole 900.

As seen in FIG. 11, the cartridge body 402 includes the first compliant puncture feature 1002-A, the second compliant puncture feature 1002-B, and a fixed puncture feature 1106. As previously discussed, the first and second compliant puncture features 1002-A, 1002-B are connected with, and/or form a part of the first containment capsule 610 and are configured to deflect in order to selectively release the contents of the first containment capsule 610. The fixed puncture feature 1106 is connected with, and/or forms a part of the second containment capsule 612. The fixed puncture feature 1106 is not as flexible or compliant as are the first and second compliant puncture features 1002-A, 1002-B. Thus, the fixed puncture feature 1106 is not deflected in order to selectively release the contents of second containment capsule 612, rather, the second containment capsule 612 is compressed onto the fixed puncture feature 1106 to thereby release the contents of the second containment capsule 612.

Each of the first and second compliant puncture features 1002-A, 1002-B and the fixed puncture feature 1106 includes at least one penetrator 1108. The penetrator 1108 can comprise a variety of shapes and sizes, and can be configured to penetrate a film or membrane to release the contents of at least a portion of a containment capsule by allowing the contents to transit through one or more holes created in the film or membrane by the penetrator. In some embodiments, the penetrator 1108 can comprise a pointed member such as one or multiple cones. In the embodiment depicted in FIG. 11, the penetrator 1108 comprises a single cone for each of the first and second compliant puncture features 1002-A, 1002-B, and a plurality of cones for the fixed puncture feature 1106. The plurality of cones of the penetrator 1108 of the fixed puncture feature 1106 are arranged around an outlet 1110 that fluidically couples to the first fluid channel 614.

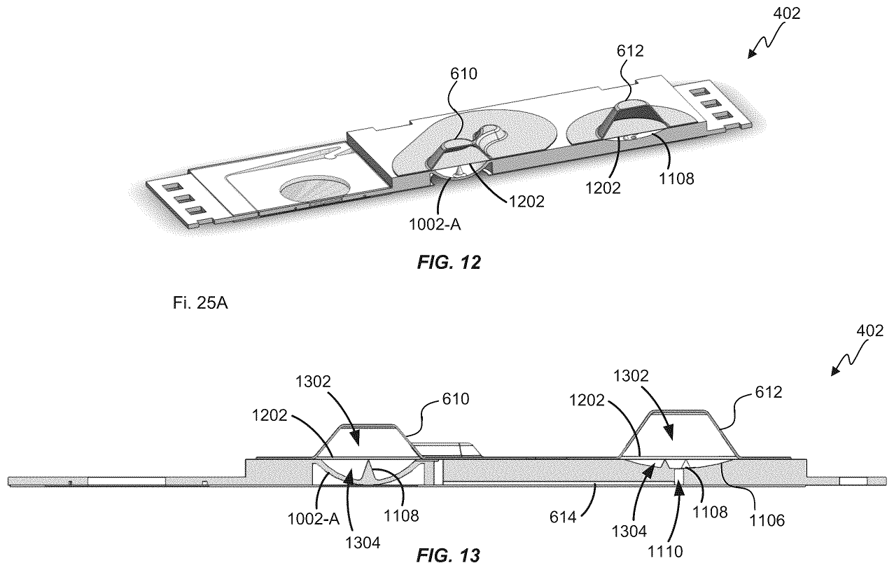

With reference now to FIG. 12, perspective section view of one embodiment of the cartridge body 402 is shown. As seen, the cartridge body 402 includes the first containment capsule 610 and the second containment capsule 612. Each of the first containment capsule 610 and the second containment capsule 612 are divided into multiple chambers by foil 1202, also referred to herein as dividing membrane 1202. The foil 1202 can be positioned within each of the first containment capsule 610 and the second containment capsule 612 such that the foil 1202 and/or a portion the foil 1202 can be punctured by the penetrator 1108 of the puncture features 1002-A, 1002-B, 1106 of the containment capsule.

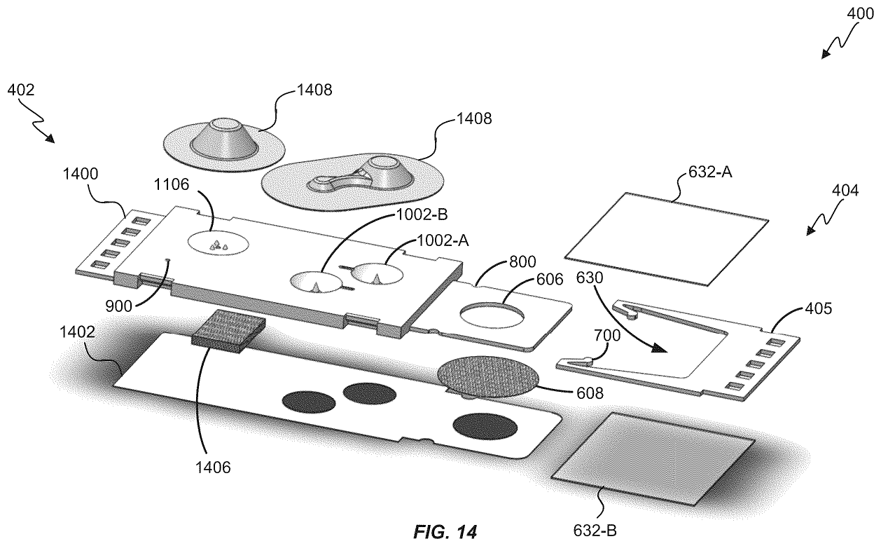

With reference now to FIG. 13, a side section view of one embodiment of the cartridge body 402 is shown. FIG. 13 is a side section view of this section shown in perspective in FIG. 12. As seen the foil 1202 divides each of the first containment capsule 610 and the second containment capsule 612 into multiple chambers. Specifically, a foil divides each containment capsule into a first chamber 1302 containing the contents of that containment capsule 610, 612, and a second chamber 1304 including the puncture features 1002-A, 1002-B, 1106 of the containment capsule 610, 612. Puncture feature 1002-B is depicted in FIG. 11 and FIG. 14.

For example, the first containment capsule 610 includes two compliant puncture features 1002-A, 1002-B. A piece of foil isolates each of these compliant puncture features 1002-A, 1002-B from first chamber 1302. The first chamber 1302 contains the contents of the first containment capsule 610, and specifically, the first chamber 1302 contains the dry reagents of the first containment capsule 610.

As each of the compliant puncture features 1002-A, 1002-B of the first containment capsule 610 are separate, puncturing the foil 1202 separating the first chamber 1302 from the second chamber 1304, which second chamber 1304 includes one of the compliant puncture features 1002-A, 1002-B only fluidly connects that one of the compliant puncture features 1002-A, 1002-B with the first chamber 1302 and thus with the contents of the first chamber 1302. Specifically, manipulation of the first compliant puncture feature 1002-A to puncture the membrane separating the first chamber 1302 from the first compliant puncture feature 1002-A fluidly connects the second chamber 1304 containing the first compliant puncture feature 1002-A to the first chamber 1302 of the first containment capsule 610, and does not connect the second chamber 1304 containing the second compliant puncture feature 1002-B to the first chamber 1302 of the first containment capsule 610. Similarly, manipulation of the second compliant puncture feature 1002-B to puncture the membrane separating the first chamber 1302 from the second compliant puncture feature 1002-B fluidly connects the second chamber 1304 containing the second compliant puncture feature 1002-B to the first chamber 1302 of the first containment capsule 610, and does not connect the second chamber 1304 containing the first compliant puncture feature 1002-A to the first chamber 1302 of the first containment capsule 610.

Thus, in some embodiments, the foil 1202 of the first containment capsule includes a first foil 1202-A separating the first chamber 1302 of the first containment capsule 610 from the second chamber 1304 containing the first compliant puncture feature 1002-A, and a second foil 1202-B separating the first chamber 1302 of the first containment capsule 610 from the second chamber 1304 containing the second compliant puncture feature 1002-B.

In contrast to the first containment capsule 610, the second containment capsule only includes a single, fixed puncture feature 1106, and thus includes only a single membrane 1202 dividing the second containment capsule 612 into the first chamber 1302 and the second chamber 1304. In some embodiments, the first chamber 1302 of the second containment capsule can include reagents, and specifically can include one or multiple aqueous reagents.

In some embodiments, each of the first fluid channel 614 and the second fluid channel 616 connect with second chambers 1304 and only are fluidly coupled with the first chambers 1302 after the puncturing of the foil 1202. Specifically, the first fluid channel 614 is fluidically coupled to the second chamber 1304 of the second containment capsule 612, and is also fluidically coupled to the second chamber 1304 containing the first compliant puncture feature 1002-A of the first containment capsule 610. The second fluid channel 616 is fluidically coupled to the second chamber 1304 containing the second compliant puncture feature 1002-B of the first containment capsule 610. Due to this architecture, both the foil 1202 of the second containment capsule 612 and the foil 1202-A of the first containment capsule 610 are punctured to allow ingress of aqueous reagents from the second containment capsule 612 into the first containment capsule 610 such that the aqueous reagents and the dry reagents mix. After the mixing of the reagents, the foil 1202-B is punctured to fluidically connect the first chamber 1302 of the first containment capsule to the second fluid channel 616, whereby the mixed reagents can flow into the reaction chamber 606.

In some embodiments, and to enable such manipulation of the cartridge body 402, the manipulation module 206 can include one or more actuators that can selectably manipulate the puncture features 1002-A, 1002-B, 1106 and/or their associated containment capsule 610, 612. In some embodiments, the manipulation module 206 can include a first actuator configured to manipulate the first compliant puncture feature 1002-A to puncture foil 1202-A, a second actuator configured to manipulate the second compliant puncture feature 1002-B to puncture foil 1202-B, and a third actuator configured to manipulate the second containment capsule 612 to puncture foil 1202 of the second containment capsule 612. In some embodiments, the first containment capsule 610 can be manipulated by a fourth actuator to cause the contents of the first containment capsule 610 to pass through the second fluid channel 616 into the reaction chamber 606.

In some embodiments, the processor 210 can be configured to control the manipulation module 206, and specifically to control the actuator(s) to first cause the mixing of the aqueous reagents and the dry reagents, and then to move the mixed reagents through the second fluid channel 616 and into the reaction chamber 606. In some embodiments, the processor 210 can be configured to control the actuator(s) to mix the aqueous reagents and the dry reagents; and to control the actuator engaging the second compliant puncture feature 1002-B to puncture film 1202-B and/or to move the mixed reagents through the second fluidic channel 616 into the reaction chamber 606.

Specifically, the processor 210 can control the first actuator to manipulate the first compliant puncture feature 1002-A to puncture foil 1202-A to fluidly couple the first chamber 1302 of the first containment capsule 610 with the first fluid channel 614, and can control the third actuator to compress the second containment capsule 612 to puncture the foil 1202 of the second containment capsule 612 to fluidly couple the first chamber 1302 of the second containment capsule 612 with the first fluid channel 614. The processor 210 can further control the third actuator to further compress the second containment capsule 612 to move the aqueous reagents from the second containment capsule 612 to mix with the dry reagents in the first chamber 1302 of the first containment capsule 610. The processor 210 can further control the second actuator to manipulate the second compliant puncture feature 1002-B to puncture foil 1202-B to fluidly couple the first chamber 1302 of the first containment capsule 610 with the second fluid channel 616 and the reaction chamber 606. The processor 210 can further control the fourth actuator to compress the first containment capsule 610 to cause the mixed reagents to fill the reaction chamber 606. In some embodiments, and as the fourth actuator compresses the first containment capsule 610, the third actuator can maintain the second containment capsule 612 in a compressed state such that the mixed reagents from the first containment capsule 610 do not flow back into the second containment capsule 612.

With reference now to FIG. 14, an exploded perspective view of the sampling unit 400. The sampling unit 400 includes the cartridge body 402 that includes a fluidic chip 1400 and a chip laminate 1402. In some embodiments, the fluidic chip 1400 can contain the fluid channels 614,616, 1102, chambers 1104, the puncture features 1002-A, 1002-B, 1106, and at least a portion of the reaction chamber 606. The fluidic chip 1400 can comprise polypropylene, polycarbonate, or the like. The chip laminate 1402 can couple to the fluidic chip 1400 to close the back 604 of the cartridge body 402, and specifically to close the fluid channels 614, 616, and the chambers 1104.

In some embodiments, the fluidic chip 1400 can be manufactured, one or multiple absorptive pads 1406 can inserted into one or multiple of the overflow chambers 1104, and specifically can be inserted into the second overflow chamber 1104-B. In some embodiments, the one or more multiple absorptive pads 1406 can be placed in the second overflow chamber 1105-B to allow overflow into the first overflow chamber 1104-A without wicking that may be caused if the one or more absorptive pads 1406 were placed in the first overflow chamber 1104-A. In some embodiments, this wicking may unnecessarily draw reagents out of the reaction chamber 606, which may inhibit and/or interfere with the nucleic acid amplification process and/or which may remove target analyte from the reaction chamber 606. In some embodiments, the one or multiple absorptive pads 1406 can prevent reagents from exiting the cartridge body 402.

The filter membrane 608 can be positioned over the reaction chamber 606, and the chip laminate 1402 can then be adhered to the fluidic chip 1400, thereby connecting the filter membrane 608 to the cartridge body 402, enclosing the one or multiple absorptive pads 1406 in one or multiple of the overflow chambers 1104, and closing the back 604 of the cartridge body 402. Capsules 1408 can be connected to the fluidic chip 1400, thereby forming the first and second containment capsules 610, 612.

In some embodiments, the cartridge receiver can be manufactured by connecting the first and second sealing members 632-A, 632-B to the body 405. In the embodiment depicted in FIG. 14, and in contrast to the embodiment of FIG. 6, the sealing members 632 are coupled to the exterior surface of the body 405, thereby defining the receptacle 630. In some embodiments, adhesive can be applied to the first and second sealing members 632-A, 632-B either before or after connecting the sealing members 632-A, 632-B to the body 405.

With reference to FIG. 15, a front view of one embodiment of the sampling unit 400 is shown. In FIG. 15, the cartridge body 402 is transparent so that otherwise hidden features are visible. As seen, the cartridge body 402 includes the first fluid channel 614 connecting the first containment capsule 610 to the second containment capsule 612, and the second fluid channel 616 connecting the first containment capsule 610 to the reaction chamber 606. The cartridge body 402 further includes exit channels 1102 and the overflow chambers 1104. Specifically, the cartridge body 402 shows the first exit channel 1102-A, the second exit channel 1102-B, and the third exit channel 1102-C, and the first overflow chamber 1104-A, the second overflow chamber 1104-B, and the third overflow chamber 1104-C. as seen, the first exit channel 1102-A connects the first overflow chamber 1104-A to the reaction chamber 606, the second exit channel 1102-B connects the first overflow chamber 1104-A to the second overflow chamber 1104-B, and the third exit channel 1102-C connects the second overflow chamber 1104-B to the third overflow chamber 1104-C.

In some embodiments, one, some, or all of the fluid channels 614, 616, 1102 can include one or several features configured to directionally limit flow of fluid therethrough. In some embodiments, for example, one, some, or all of the fluid channels 614, 614, 1102 can include a one-way valve configured to allow fluid flow in a desired direction and to prevent fluid flow in an opposite direction. In some embodiments, for example, a one-way valve can be positioned in the first fluid channel 614 between the first containment capsule 610 and the second containment capsule 612, and/or a one-way valve can be positioned in the second fluid channel 616 between the first containment capsule 610 and the reaction chamber 606. In some embodiments, a one-way valve in the second fluid channel 616 can prevent the movement of fluid into and/or out of the reaction chamber 606 via the second fluid channel during the nucleic acid amplification process, and specifically during thermocycling.

The cartridge body 402 further includes the first foil 1202-A connecting the first chamber 1302 of the first containment capsule 610 to the second chamber 1304 of the first containment capsule 610, which second chamber 1304 of the first containment capsule 610 includes the first compliant puncture feature 1002-A and is fluidly connected to the first fluid channel 614. The cartridge body 402 further includes the second foil 1202-B connecting the first chamber 1302 of the first containment capsule 610 to the second chamber 1304 of the first containment capsule 610, which second chamber 1304 of the first containment capsule 610 includes a second compliant puncture feature 1002-B and is fluidly connected to the second fluid channel 616. The cartridge body 402 additionally includes the foil 1202 separating the first chamber 1302 of the second containment capsule 612 from the second chamber 1304 of the second containment capsule 612, which second chamber 1304 includes the fixed puncture feature 1106 and is fluidly connected to the first fluid channel 614.

In some embodiments, the sampling unit 400 can be used in connection with the fluid sampling and analysis system 102, and in some embodiments, the sampling unit 400 can be used independent of the fluid sampling and analysis system 102. In some embodiments, for example, the sampling unit 400 can be configured to receive a sample on the cartridge body 402, and specifically within the reaction chamber 606 of the cartridge body 402. In some embodiments, the sample can be received on the SCS, which can comprise a filter membrane configured to capture the sample from a fluid passing through the filter membrane, or the SCS can be a substrate and/or feature configured to receive and/or hold a sample. In some embodiments, for example, the sample can comprise a drop of bodily fluid, such as a drop of blood, and the SCS and/or reaction chamber can merely receive and/or hold that drop of blood. In such an embodiment, the cartridge body 402 can be received within the receptacle 630 of the cartridge receiver 404, the reaction chamber 606, including the SCS 608 can be enclosed, and a nucleic acid amplification process can be performed on the enclosed reaction chamber 606 including on the SCS 608. In some embodiments, the sample can comprise a drop of bodily fluid, such as a drop of blood, and the sample is placed or conveyed directly into the reaction chamber without the presence of an SCS.

In some embodiments, the sampling unit 400 can be utilized in the collection of a sample, and in the analysis of that sample from, for example, one or several humans, animals, or the like. Regardless how the sample is collected and regardless of whether an SCS is utilized, and after collection of the sample, the cartridge body 402 can be received within receptacle of the cartridge receiver 404, and the nucleic acid amplification process can be performed on the reaction chamber and on the SCS that is included in the reaction chamber. In some embodiments, this can include positioning the reaction chamber 606 and the SCS in a first position with respect to the cartridge receiver 404 to collect the sample, and positioning the reaction chamber 606 and the SCS 608 is a second position with respect to the cartridge receiver 404 to perform nucleic acid amplification on the sample and to analyze the sample.

In one embodiment, a human blows air, using a disposable mouthpiece for example, directly into the fluid transport module 208 such that at least some of the microbes such as bacteria, viruses, etc. that are entrained in the air exhaled from the person's respiratory tract are captured by the SCS. In one embodiment the sampling unit 400 measures the volume of air that has been blown through the fluid transport module 208 until a predetermined volume of air has passed through the SCS, at which point the fluid transport module 208 closes and does not allow further air to pass through. Thereafter, the sampling unit 400 proceeds with analyzing the sample as disclosed herein. In some embodiments the fluid transport module 208 contains a one-way valve such that air cannot flow toward the mouthpiece. In some embodiments the disposable mouthpiece can be coupled to the fluid transport module 208 directly at the SCS and/or in such a way that minimal or no cross contamination between human subjects occurs with respect to surfaces within the fluid transport module 208 prior to the point where the exhaled air hits the SCS. In some embodiments, once the result of analysis are obtained from a human breath sample, the communications module 214 transmits results to a gating system/device 106, such that the person who provided the sample is then allowed or not allowed, depending on the result, to exit the premises or pass through an access portal, such as to then board a commercial aircraft or enter a space containing other humans who have already been screened and cleared for passage. Alternatively, the sampling and analysis system can be placed inside a chamber that a human enters and remains in for a period of time such that the system can be used as a noninvasive diagnostic device. The sampling and analysis system is therefore useful for not only human diagnostic applications, but also for veterinary and livestock applications such as monitoring the health and disease state of a single animal or a population of animals.

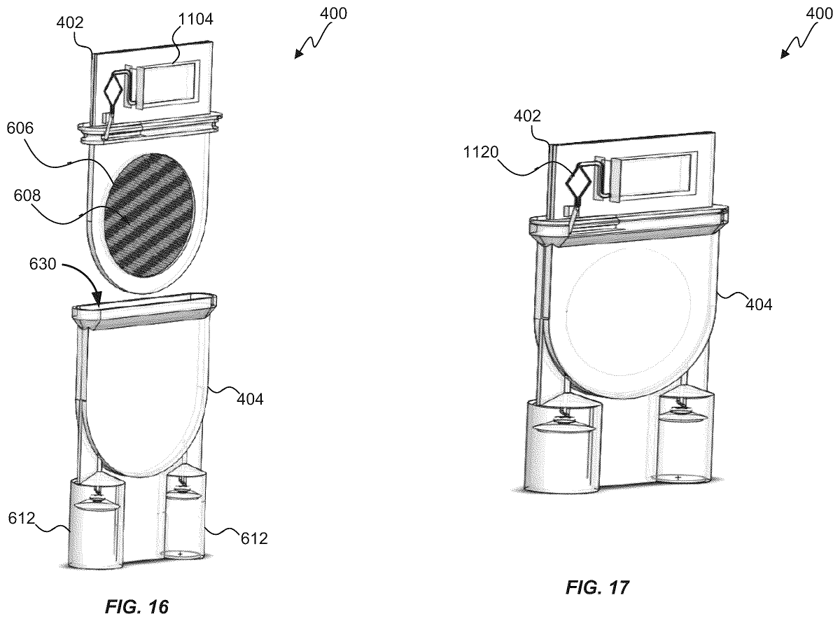

With reference now to FIGS. 16 and 17, perspective view of another embodiment of a sampling unit 400 is shown. The sampling unit 400 includes the cartridge body 402 and the cartridge receiver 404. The cartridge body 402 includes an amplification chamber 1120, the SCS 608, and an overflow chamber 1104. A plunger pushes into containment capsule 612 to convey aqueous rinse buffer over the SCS 608 after sample capture. The buffer removes analyte from the SCS and the mixture is conveyed to a channel junction. Nucleic acid amplification reagents are conveyed from the other containment capsule 612 to the channel junction, at which point the Nucleic acid amplification reagents and the rinse buffer containing analyte are conveyed to the amplification chamber 1120. In some embodiments, the amplification chamber 1120 can be transparent to allow the excitation of the contents of the amplification chamber 1120 and/or the detecting of fluorescence emitted by the contents of the amplification chamber 1120.

The sampling unit 400, and specifically the cartridge receiver 404 can define the receptacle 630 configured to receive a portion of the cartridge body 402, and specifically configured to receive the reaction chamber 606 and/or the SCS 608. The sampling unit 400, and specifically the cartridge receiver 404 can include one or several second containment capsules 612 that can, in some embodiments, hold one or several aqueous reagents.

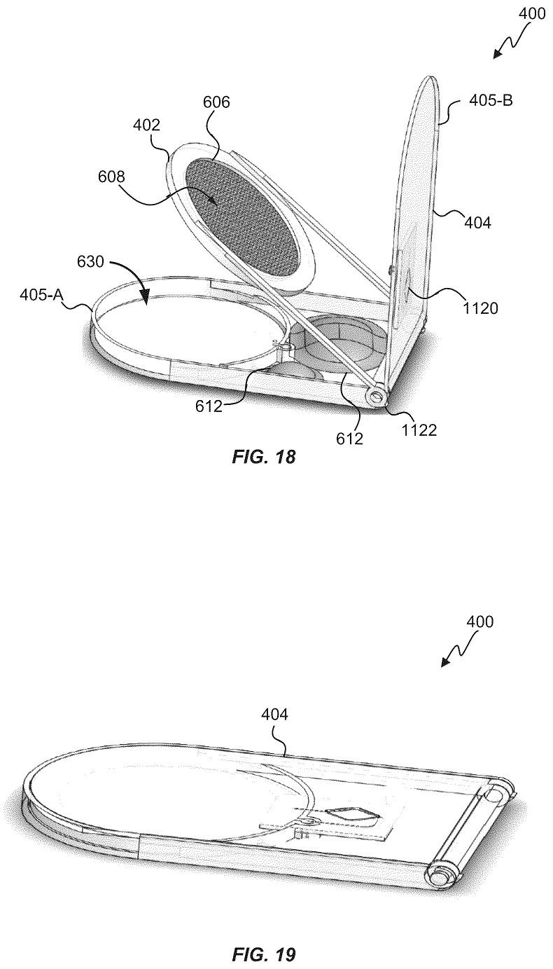

With reference now to FIGS. 18 and 19, a perspective view of a hinged embodiment of a sampling unit 400 is shown. The sampling unit 400 includes the cartridge body 402 and the cartridge receiver 404. The cartridge body 402 includes the SCS 608. The cartridge receiver 404 includes a first body piece 405-A and a second body piece 405-B that are connected to the cartridge body 402 via a hinge 1122. In such an embodiment, the first body piece 405-A and the second body piece 405-B can pivot about the hinge 1122 such that the cartridge body 402, and specifically the SCS 608 is between the first body piece 405-A and the second body piece 405-B. In such a position, the SCS 608 can be received within the receptacle 630 of the cartridge receiver 606.

The sampling unit 400, and in some embodiments, the cartridge receiver 404 can include one or several containment capsules which can contain one or several reagents, and specifically, one or several nucleic acid amplification reagents. In some embodiments, these containment capsules can include one or several first containment capsules 610 containing one or several dry reagents, and/or one or several second containment capsules 612 containing one or several aqueous reagents. In the embodiment shown in FIGS. 18 and 19, the cartridge body includes a plurality of second containment capsules 612. In some embodiments, the contents of the capsules are mixed, conveyed through the SCS to capture analyte, and further conveyed to the amplification chamber 1120 in which the nucleic acid amplification process and analysis can be performed.

In some embodiments, the sampling unit 400 shown in FIGS. 18 and 19 can be moved to a first position in which the SCS 608 is exposed for sample collection, and can be moved to a second position, such as shown in FIG. 19, in which the SCS 608 is enclosed within the receptacle 630 by the first and second body pieces 405-A, 405-B. While the SCS 608 is enclosed within the receptacle 630, the nucleic acid amplification process and analysis can be performed.

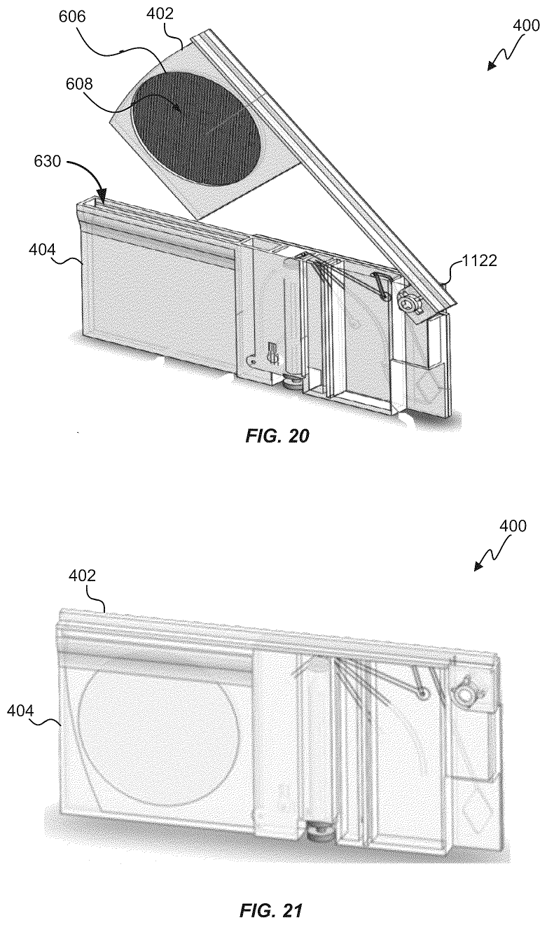

With reference now to FIGS. 20 and 21, a perspective view of another hinged embodiment of a sampling unit 400 is shown. The sampling unit 400 includes the cartridge body 402 and the cartridge receiver 404. The cartridge body 402 includes the reaction chamber 606 including the SCS 608. The cartridge receiver 404 can be connected to the cartridge body 402 via a hinge 1122. In such an embodiment, the cartridge body 402 can be moved to a first position in which the SCS 608 and/or the reaction chamber 606 is exposed for sample collection and/or for receiving a sample, and a second position in which the SCS 608 and/or the reaction chamber 606 is contained within the receptacle 630 of the cartridge receiver 404.

In some embodiments, the sampling unit 400 shown in FIG. 20 can be moved to the first position in which the SCS 608 is exposed for sample collection, and can be moved to a second position, such as shown in FIG. 21, in which the SCS 608 is enclosed within the receptacle 630. While the SCS 608 is enclosed within the receptacle 630, the nucleic acid amplification process and analysis can be performed with reagents contained in, for example, one or several containment capsules of the sampling unit 400.

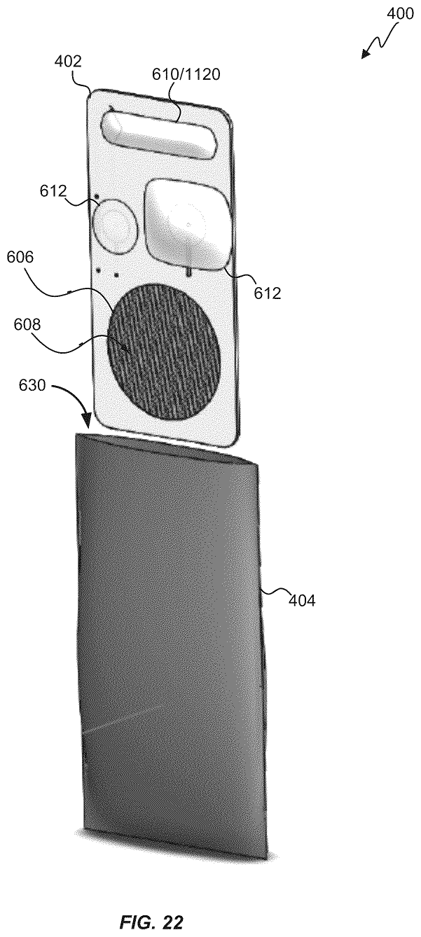

With reference now to FIG. 22, a perspective view of another embodiment of a sampling unit 400 is shown. The sampling unit 400 includes the cartridge body 402 and the cartridge receiver 404. The cartridge body 402 includes the reaction chamber 606 including the SCS 608. The cartridge body 402 further includes a second containment capsule 612. In some embodiments, after sample capture, the contents of the containment capsules 612 are mixed and conveyed into the reaction chamber 606 to saturate the SCS 608. The cartridge body 402 is then inserted into the cartridge receiver 404 and a watertight perimeter is heat staked around the reaction chamber 608, thereby sealing the cartridge receiver 404 to enclose the reaction chamber 608. In some embodiments, the cartridge receiver 404 can be transparent to allow the excitation of the contents of the reaction chamber 606 and/or the detecting of fluorescence emitted by the contents of the reaction chamber 606 during thermocycling.

The cartridge receiver 404 can define the receptacle 630 configured to receive a portion of the cartridge body 402, and specifically configured to receive the reaction chamber 606 and/or the SCS 606. In the embodiment of FIG. 21, the cartridge receiver 404 comprises a pouch configured to receive at least the reaction chamber 606 and/or the SCS 608 to thereby enclose the reaction chamber 606 including the SCS 608.

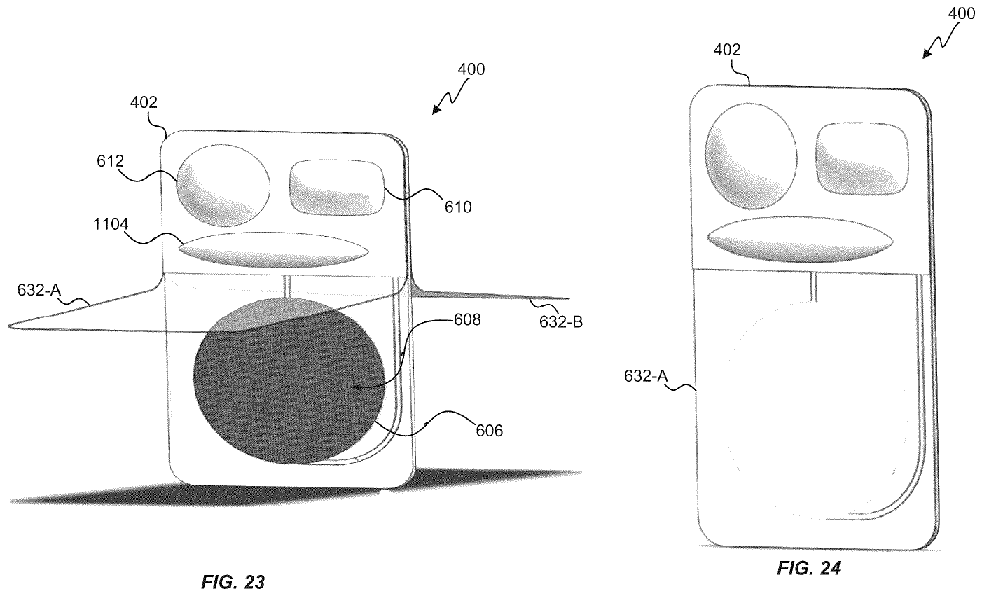

With reference now to FIGS. 23 and 24, a perspective view of another embodiment of a unitary sampling unit 400 is shown. The cartridge body 402 includes the reaction chamber 606 including the SCS 608. The cartridge body 402 further includes a first containment capsule 610 holding the dry reagents, and the second containment capsules 612 hold one or several aqueous reagents. The cartridge body 402 further includes the overflow chamber 1104. The first and second containment capsules 610, 612 are fluidly connected to each other and to the reaction chamber 606 such that the aqueous reagents pass to the first containment capsule 610, where they mix with the dry reagents before passing to the reaction chamber 606. Any excess mixed reagents can exit the reaction chamber 606 into the overflow chamber 1104.

The sampling unit 400 includes the cartridge body 402 including moveable sealing members 632. The sealing members 632 can be coupled to the cartridge body 402 such that the sealing members are movable from a first position in which the reaction chamber 606 is open for receiving a sample, and a second position in which the reaction chamber 606 is enclosed. In some embodiments, the sealing members 632 can be moved from the first position, as shown in FIG. 23 to the second position, as shown in FIG. 24, and can be sealed around the reaction chamber 606, thereby enclosing the reaction chamber 606. After the reaction chamber 606 has been enclosed and sealed, the nucleic acid amplification process can be performed in the reaction chamber 606.

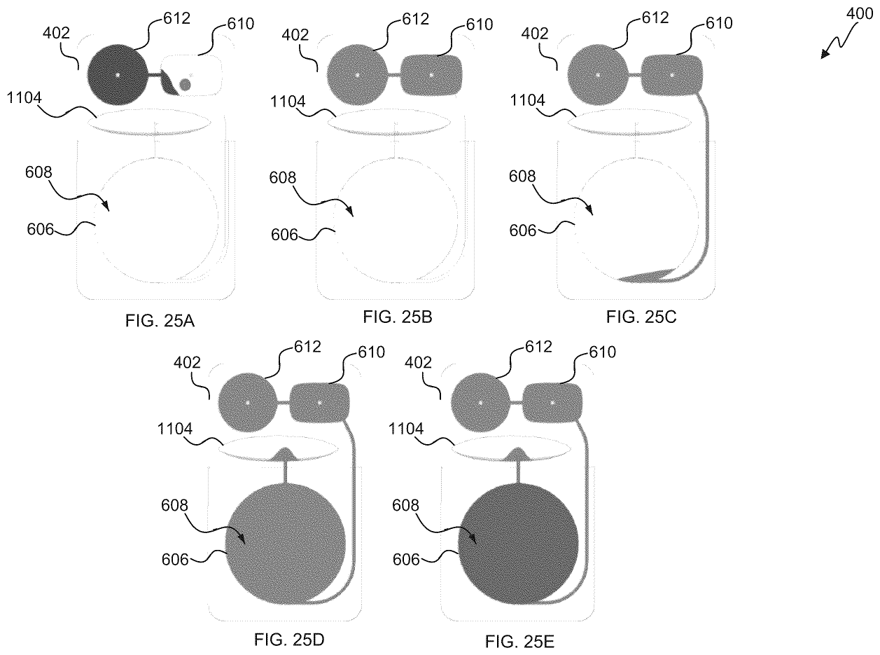

FIG. 25A-FIG. 25E are a depiction of a process for direct nucleic acid amplification and analysis, or in other words, nucleic acid amplification and analysis performed on a reaction chamber 606 containing an SCS 608. FIG. 25A-FIG. 25E depict a cartridge body 402 including the reaction chamber 606 with an SCS 608, first and second containment capsule 610, 612, and overflow chamber 1104. As seen in FIG. 25A, the aqueous reagents of the second containment capsule 612 are fluidly coupled with the dry reagents of the first containment capsule 610, and as shown in FIG. 25B, these reagents are mixed. The reaction chamber 606 is then filled with the mixed reagents as shown in FIG. 25C until the reaction chamber 606 is fully filed as depicted in FIG. 25D. The filling of the reaction chamber 606 with the mixed reagents can immerse the SCS 608 in the reagents. In some embodiments, some amount of mixed reagents can flow from the reaction chamber 606 into the overflow chamber 1104. As depicted in FIG. 25E, the nucleic acid amplification and analysis process is performed directly on the reaction chamber 606 and the scs 608.

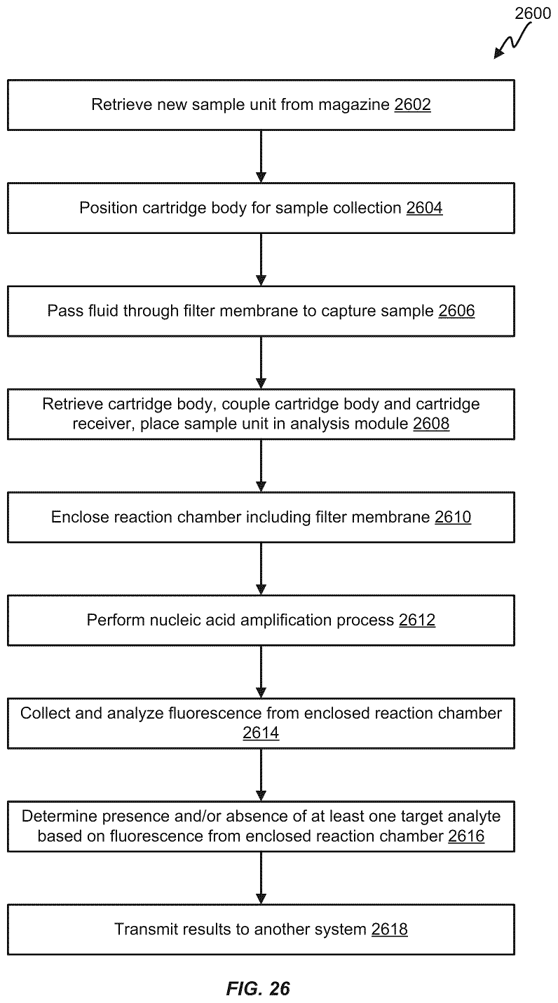

With reference now to FIG. 26, a flowchart illustrating one embodiment of a process 2600 for collecting and analyzing a sample is shown. The process can be performed by all or portions of the fluid sampling and analysis system 102. The process 2600 begins at block wherein a new sample unit 400 is retrieved from the magazine 202. In some embodiments, retrieving a new sample unit 400 from the magazine 202 can include the processor 210 checking whether a new sample unit 400 is available in the magazine 202. If a new sample unit 400 is not available in the magazine 202, then the processor 210 can generate an alert which can be transmitted, for example, via the communications module 214. This alert can indicate that the magazine 202 should be refilled. The processor 210 can also transmit the current new/spent count of sample units upon request and/or send alerts that the new sample units will all be consumed at a certain time and date based on the programmed rate of sampling.

Alternatively, if it is determined that there is a new sample unit 400 available in the magazine 202, the processor 210 can direct the manipulation module to remove a sample unit 400 containing a collected sample from the fluid transport module, and retrieve the new sample unit 400 from the magazine 202. The manipulation module 206 can execute the control signals received from the processor 210 and can retrieve the new sample unit 400 from the magazine 202.

At block 2604 the cartridge body 402 of the sampling unit 400 can be positioned for sample collection. In some embodiments, this can include preparing the sampling unit 400 and specifically the cartridge body 402 for passing a fluid through the reaction chamber 606 and specifically through the filter membrane 608. In some embodiments, preparing the sampling unit can include separating the cartridge body 402 from the cartridge receiver 404 such that the reaction chamber 606 is outside of the receptacle 630 of the cartridge receiver 404. In some embodiments, preparing the sampling unit 400 can include unsealing the reaction chamber 606 from a configuration in which the filter membrane 608 has not been exposed to air outside of the reaction chamber 606 and/or sampling unit since it was manufactured, and therefore is not contaminated with any microbes prior to capturing analyte. After the sampling unit has been prepared, the sampling unit 400, and specifically the cartridge body 402 can be inserted into and/or coupled to the fluid transport module 208 such that the fluid transport module 208 can move fluid through the reaction chamber 606 in the filter membrane 608 to capture a sample.

In some embodiments, positioning the cartridge body for sample collection can be controlled by the processor 210, which processor 210 can generate control signals to cause the manipulation module 206 to prepare the sample unit for sample collection and to cause the manipulation module to position the sampling unit 400, and specifically the cartridge body 402 for sample collection. The manipulation module 206 can execute the control signals received from the processor 210 and can prepare the sample unit 400 for sample collection and can position the sample unit 400 in or coupled to the fluid transport module 208 so that fluid can be moved through the reaction chamber 606 and the filter membrane 608.

At block 2606 fluid is passed through the reaction chamber 606 and specifically through the filter membrane 608 to capture a sample. In some embodiments, the fluid can be conveyed through the reaction chamber 606 and specifically through the filter membrane 608 by the fluid transport module 208. In some embodiments, this fluid can be air, water, sewage, or the like.

In some embodiments, the processor 210 can control the fluid transport module 208 to move fluid through the reaction chamber 606 and specifically through the filter membrane 608 to capture the sample. In some embodiments, the processor 210 can control the fluid transport module 208 to move fluid through the filter membrane 608 at between approximately 0.1 LPM/mm2 of the filter membrane 608 and 10 LPM/mm2 of the filter membrane 608. In some embodiments, the processor 210 can control the fluid transport module 208 to move fluid through the filter membrane 608 at between approximately 1 LPM/mm2 of the filter membrane 608 and 5 LPM/mm2 of the filter membrane 608. In some embodiments, the processor 210 can control the fluid transport module 208 to move fluid through the filter membrane 608 at a flowrate between 0.1 LPM/mm2 of the filter membrane 608 and 10 LPM/mm2 of the filter membrane 608 such that less than 50 Db is generated at a distance of 36 inches from the sampling and analysis system 102. In some embodiments, the processor 210 can control the fluid transfer module 208 such that each of the collected samples captures analyte from between approximately 500 L of air through the filter membrane 608 and approximately 100,000 L of air through the filter membrane 608.

In some embodiments, the sampling and analysis system 102 can commence air sampling upon receiving a signal. In some embodiments, this signal can be initiated in response to the carbon dioxide level inside a building rising above a threshold level. In some embodiments, this signal can be initiated manually from a remote location. In some embodiments, the sampling and analysis system 102 performs air sampling during predetermined hours that correspond to times of human occupancy inside a building above a certain density of humans per square foot.

A block 2608, and upon completion of sample capture, the cartridge body 602 is retrieved from the fluid transport module 208, the cartridge body 402 and the cartridge receiver 404 are coupled, and the sampling unit 400 is placed in the analysis module 204. In some embodiments, the processor 210 can generate control signals directing the manipulation module 206 to retrieve the cartridge body 402 from the fluid transport module 208, to couple the cartridge body 402 and the cartridge receiver 404, and to place the sampling unit 400 in the analysis module 204. The manipulation module 206 can receive these control signals and can execute according to the control signals.

In some embodiments the analysis module 204 commences thermocycling on a sample unit 400 containing analyte simultaneous with a new sample unit 400 being placed by the manipulation module 206 into the fluid transport module 208. In other words, the biosensor 102 is capable of capturing a new sample while a previously captured sample is still in the process of being analyzed by nucleic acid amplification. In some embodiments the processor 210 receives results from the analysis module indicating a certain pattern of microbes are present in the sampled environment. Based on these results, the processor 210 instructs the manipulation module 206 to select a certain type of new sample unit 400 from the magazine 202. For example, if an analysis of a first sample unit 400 indicates the presence of a coronavirus using PCR primers that are generic for all types of coronavirus, the processor 200 can instruct the manipulation module 206 to select a specific new sample unit 400 from the magazine 200 that is loaded with PCR primers capable of distinguishing between SARS-CoV-2 (cause of the COVID-19 pandemic), SARS-CoV (cause of the 2002-2004 SARS outbreak) and MERS-CoV (cause of the 2012 MERS outbreak). Having a magazine 202 containing sample units containing different PCR reagents specific for different microbes, including reagents that broadly detect certain classes of pathogen, and reagents that detect specific genera, species or strains of microbes within a certain broad class allows the biosensor to broadly scan a fluid such as air for certain types of pathogen and then rapidly determine the exact type of pathogen without the need for human intervention. The advantage to operating the biosensor 102 this way is that it can avoid missing certain types of pathogen by only running PCR reagents specific to a certain species or strain of microbe.

In some embodiments, and upon removing the cartridge body 402 from the fluid transport module 208, a new cartridge body 402 can be retrieved from the magazine 202 and positioned to collect a sample if an additional sample is desired. Thus, in some embodiments, a new sampling unit 400 can be retrieved from the magazine 202 and the new sampling unit 400 can be positioned to collect a new sample by passing fluid through the reaction chamber 606 and the filter membrane 608 of the cartridge body 402 of the new sampling unit 400.

In some embodiments, coupling the cartridge body 402 and the cartridge receiver can include inserting the cartridge body 402 into the receptacle 630 of the cartridge receiver 404. In some embodiments, the cartridge body 402 can be inserted into the receptacle 630 of the cartridge receiver 404 until the reaction chamber 606 of the cartridge body 402 is fully enclosed within the cartridge receiver 404, and specifically is fully enclosed within the receptacle 630 of the cartridge receiver 404. In some embodiments, when the cartridge body 402 is fully inserted into the receptacle 630 of the cartridge receiver 404, one or multiple locking features 700 of one of the cartridge body 402 and the cartridge receiver 404 can engage with one or multiple meeting features 800 of the other of the cartridge body 402 and the cartridge receiver 404 to thereby secure the cartridge body 402 to the cartridge receiver 404.

At block 2610 the reaction chamber 606 is enclosed. In some embodiments, enclosing the reaction chamber likewise encloses the filter membrane 608 within the reaction chamber 606. In some embodiments, inserting the cartridge body 402 into the receptacle 630 of the cartridge receiver 404 can enclose the reaction chamber 606 of the cartridge body 402. In some embodiments, the reaction chamber 606 is enclosed by the sealer 306 sealing the sealing members 632 of the cartridge receiver 404 to the cartridge body 402. In some embodiments, the first sealing member 632-A can be sealed to the front 602 of the cartridge body 402 around the perimeter of the reaction chamber 606. Likewise, in some embodiments, the second sealing member 632-B can be sealed to the back 604 of the cartridge body 402 around the perimeter of the reaction chamber 606. In some embodiments, the sealer 306 can seal the sealing members 632 to the cartridge body via, for example, an adhesive, a weld, a heat weld, a pressure weld, a laser weld, an ultrasonic weld, a friction weld, a solid-state weld, heat-staking, or the like.

In some embodiments, the processor 210 can control the enclosing of the reaction chamber 606 by generating and sending control signals to the manipulation module 206 to at least partially insert the cartridge body 402 into the cartridge receiver 404, and to the sealer 306 to seal the first and second sealing members 632-A, 632-B to the cartridge body 402 around the perimeter of the reaction chamber 606. In some embodiments, sealing the first and second sealing members 632-A, 632-B to the cartridge body 402 around the perimeter of the reaction chamber 606 can include sealing the first sealing member 632-A to the front 602 of the cartridge body 402 and sealing the second sealing member 632-B to the back 604 of the cartridge body 402.

At block 2612 a nucleic acid amplification process is performed on the contents of the enclosed reaction chamber 606 including on the filter membrane 608 contained in the enclosed reaction chamber 606. In some embodiments, the nucleic acid amplification process can be performed directly on the reaction chamber 606 and/or directly on the filter membrane 608. In some embodiments, performing the nucleic acid amplification reaction directly on the reaction chamber 606 and on the filter membrane can include filling the reaction chamber 606 with nucleic acid amplification reagents, and thermocycling the reaction chamber 606 and the filter membrane 608 contained in the reaction chamber 606. In some embodiments, filling the reaction chamber 606 with nucleic acid amplification reagents can include mixing aqueous nucleic acid amplification reagents with dry nucleic acid amplification reagents, and filling the mixed nucleic acid amplification reagents into the reaction chamber 606.

In some embodiments, performing the nucleic acid amplification process can include, for example, sealing the second fluid channel 616 and/or fluidic channel 1102-A subsequent to the filling of the reaction chamber 606 with reagents and before thermocycling. In some embodiments, the second fluid channel 616 and/or fluidic channel 1102-A can be sealed to prevent reagents from exiting and/or entering the reaction chamber 606 during the nucleic acid amplification process, and specifically during thermocycling.

In some embodiments, the nucleic acid application process can comprise an isothermal nucleic acid amplification process, or a non-isothermal nucleic acid amplification process. In some embodiments, the nucleic acid implication process can comprise a PCR process. In some embodiments, the nucleic acid amplification process can be performed according to control signals generated by the processor 210 by the manipulation module 206, and specifically the fluid actuators 308, and the analysis module 204, and specifically the thermocycler 302.

At block 2614 fluorescence from the enclosed reaction chamber 606 is collected and analyzed. In some embodiments, the collection and analysis of the fluorescence from the enclosed reaction chamber 606 can include the measuring of fluorescence from the enclosed reaction chamber 606. In some embodiments, the fluorescence can be collected and/or analyzed by the analysis module 204 and specifically by the optics 304. In some embodiments, the collection and analysis of fluorescence can include the application of excitation energy to the reaction chamber, and the capturing of fluorescence emitted by the reaction chamber 606 and the contents of the reaction chamber including the filter membrane 608. In some embodiments, the collection analysis of fluorescence from the enclosed reaction chamber 606 can be performed according to one or multiple control signals generated by the processor 210.

In some embodiments, and upon completion of the collecting and analyzing of fluorescence from the enclosed reaction chamber 606, the cartridge body 402 containing the analyzed sample can be ejected from the analysis module 204 and can be disposed of. In some embodiments this can include placing the used sampling unit 400 in a portion of the magazine 202 four containing used sampling units 400.

At block 2616 the presence and or absence of at least one target analyte is determined based on fluorescence collected from the enclosed reaction chamber 606. In some embodiments, this can include detecting the presence and/or absence of one or more airborne pathogens based on the fluorescence signal collected and analyzed in block 2614. In some embodiments, the presence or absence of at least one target analyte can be determined by the analysis module 204 and/or by the processor 210.

At block 2618 the results are transmitted to another system In some embodiments, these results can be electronically transmitted to another system by, for example, the communications module 214. In some embodiments, electronically transmitting the results to another system can include wired or wirelessly transmitting the results to another system such as the control system 104 and/or one or multiple of the additional systems/devices 106. In some embodiments, this other system can be at least one of a building ventilation system, an alarm, a notification system, or the like.

In some embodiments, the results can include indications of the presence or absence of at least one target analyte. This target can be one or multiple pathogens, and specifically can be one or multiple airborne pathogens. Thus, in some embodiments, the results can indicate the detected presence, absence, and/or concentration of one or more airborne pathogens in the sample.

The process 2600 can be performed once or multiple times within a time period. For example, in some embodiments, the process 2600 can continuously performed such that a sample is collected while a previously collected sample is analyzed. In some embodiments, such performance of the process 2600 can include passing air through the filter membrane 608 to capture 48 samples of analyte in a day. Each of those 48 samples can be analyzed subsequent to collection of that sample. In some embodiments, analyzing each of those 48 samples subsequent to collection of that sample can include closing the filter membrane 608 within the reaction chamber 606, performing the nucleic acid amplification reaction directly on the reaction chamber 606 and on the filter membrane 608, and measuring the fluorescent signal from the reaction chamber 606 and from the filter membrane 608. In some embodiments, the results of each of the samples can be transmitted to another system via, for example, a wired or wireless communication.

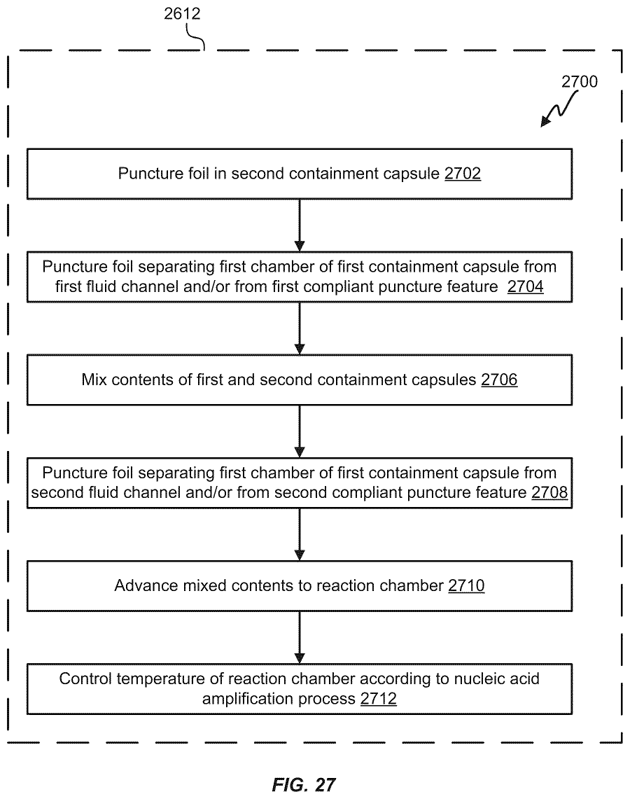

With reference to FIG. 27, flowchart illustrating one embodiment of a process 2700 for performing a nucleic acid amplification process is shown. In some embodiments, the nucleic acid amplification process can comprise at least one of: qPCR, PCR, RT-PCR, RT-qPCR, digital PCR, an isothermal amplification process, immuno-PCR, proximity ligation PCR or the like.

The process can be performed as a part of, or the place of the step of block 2612 of process 2600. The process 2700 can be performed by all or portions of the fluid sampling and analysis system 102. The process 2700 begins at block 2702, wherein foil 1202 in the second containment capsule 612 is punctured. In some embodiments, the foil 1202 in the second containment capsule 612 is punctured by the processor 210 generating control signals controlling an actuator in the manipulation module 206 to compress the second containment capsule 612 such that the foil is forced onto the at least one penetrator 1108 of the fixed penetration feature 1106. In some embodiments, puncturing the foil 1202 of the second containment capsule 612 can fluidly connect the contents of the second containment capsule 612 to the first fluid channel 614.

At block 2704, foil 1202-A separating the first chamber 1302 of the first containment capsule 610 from the first fluid channel 614 and/or from the first compliant puncture feature 1002-A can be punctured. In some embodiments, the foil 1202-A is punctured by the processor 210 generating control signals controlling an actuator in the manipulation module 206 to compress the first complaint puncture feature 1002-A to move toward the foil 1202-A until the at least one penetrator 1108 of the compliant puncture feature 1002-A punctures the foil 1202-A. In some embodiments, puncturing the foil 1202-A separating the first chamber 1302 of the first containment capsule 610 from the first fluid channel 614 and/or from the first compliant puncture feature 1002-A can fluidly connect the contents of the first chamber 1302 of the first containment capsule 610 with the contents of the second containment capsule 612.

At block 2706, the contents of the first and second containment capsules 610, 612 are mixed. In some embodiments, this can include the processor 210 controlling an actuator to compress the second containment capsule 612 to advance the contents of the second containment capsule 612 through the first fluid channel 614 and into the first chamber 1302 of the first containment capsule 610. In some embodiments, the contents of the second containment capsule 612 can comprise one or multiple aqueous reagents and the contents of the first containment capsule 610 can comprise one or multiple dry reagents. Mixing the contents of the first and second containment capsules 610, 612 can activate the dry reagents and can create a homogeneous solution that is capable of detecting one or more analytes when put through a nucleic acid amplification reaction such as thermocycling in the presence of an SCS.

At block 2708 the foil 1202-B separating the first chamber 1302 of the first containment capsule 610 from the second fluid channel 616 and/or from the second compliant puncture feature 1002-B can be punctured. In some embodiments, the foil 1202-B is punctured by the processor 210 generating control signals controlling an actuator in the manipulation module 206 to compress the second complaint puncture feature 1002-B to move toward the foil 1202-B until the at least one penetrator 1108 of the second compliant puncture feature 1002-B punctures the foil 1202-B. In some embodiments, puncturing the foil 1202-B separating the first chamber 1302 of the first containment capsule 610 from the second fluid channel 616 and/or from the second compliant puncture feature 1002-B can fluidly connect the mixed contents of the first chamber 1302 of the first containment capsule 610 with the reaction chamber 606.

At block 2710 the mixed contents are advanced from the first chamber 1302 of the first containment capsule 610 and through the second fluid channel 616 to the reaction chamber 606. In some embodiments, the mixed contents can be advanced to the reaction chamber 606 until the reaction chamber is filled with the mixed reagents, which can include nucleic acid amplification reagents, from the first chamber 1302 of the first containment capsule 610. In some embodiments, this can include the processor 210 controlling an actuator to compress the first containment capsule 610 to advance the mixed reagents of the first containment capsule 610 through the second fluid channel 616 and into the reaction chamber 606. In some embodiments, the filling of the reaction chamber 606 by the mixed reagents immerses the filter membrane 608 in the mixed reagents. In some embodiments, excess mixed reagents advanced into the reaction chamber can exit the reaction chamber 606 via the exit channels 1102 and can feel and/or partially fill the overflow chambers 1104. In some embodiments, for example, excess reagents can be absorbed by the one or multiple absorptive pads 1406 that can be inserted into one or multiple of the overflow chambers 1104. In some embodiments, the sampling unit 400 is held in a vertical position such that the reaction chamber 606 is at the bottom (the left side of FIG. 15) and the second containment capsule is at the top (the right side of FIG. 15), thereby allowing the fluidic channel 616 to fill the reaction chamber 606 from the lowest point, thereby filling the reaction chamber from the bottom up and pushing any air bubbles out the top through fluidic channel 1102-A.

At block 2712, the temperature of the reaction chamber 606 is controlled according to the particular nucleic acid amplification process being performed. In some embodiments, this can include heating the reaction chamber 606 and the contents of the reaction chamber 606 including the filter membrane 608 to a first temperature to enable reverse transcription, and then, upon completion of the reverse transcription step, cyclically heating and cooling the reaction chamber 606 and the contents of the reaction chamber 606 including the filter membrane 608 to perform a PCR reaction.

In some embodiments, the nucleic acid amplification process does not include a traditional lysis or extraction step. Rather, lysis of the analyte occurs during sample capture and during the reverse transcription phase. Thus, in some embodiments, the target nucleic acid becomes available for exposure to reagents during sample capture and during the reverse transcription phase. Advantageously, this lysis occurs at temperatures that prevent the deactivation of the reverse transcriptase. Conventional analysis methods use an extraction and/or rinsing step, such as PCR testing of nasal swabs where the swab is rinsed and discarded. The design and operation of the devices disclosed herein are based on the insight that because microbes can be relatively dilute in air compared to the high concentrations found, for example, on the interior surfaces of a human nasal passage, achieving high sensitivity from shorter sampling times is of paramount importance. This is achieved in part by building a nucleic acid amplification reaction chamber around an SCS immediately after sampling is complete and performing nucleic acid amplification directly on an SCS rather than rinsing/extracting the SCS and transferring analyte, which results in yield loss. Building a nucleic acid amplification reaction chamber around an SCS, wherein the SCS occupies greater than 40%, 50%, 60%, 70%, 80% or 90% of the total volume of the reaction chamber increases sensitivity while reducing reagent volume and thereby cost. Placement of the sampling and analysis system 102 can be indoors or outdoors, including locations such as a building lobby or other common area, airport gate, bathroom, attached to an HVAC return air duct such that air from the duct enters the fluid transport module 208, inside an HVAC system, offices, transportation vessels such as ships, aircraft, trains, subways and submarines, doctor offices, hospitals and the like.

### Example 1

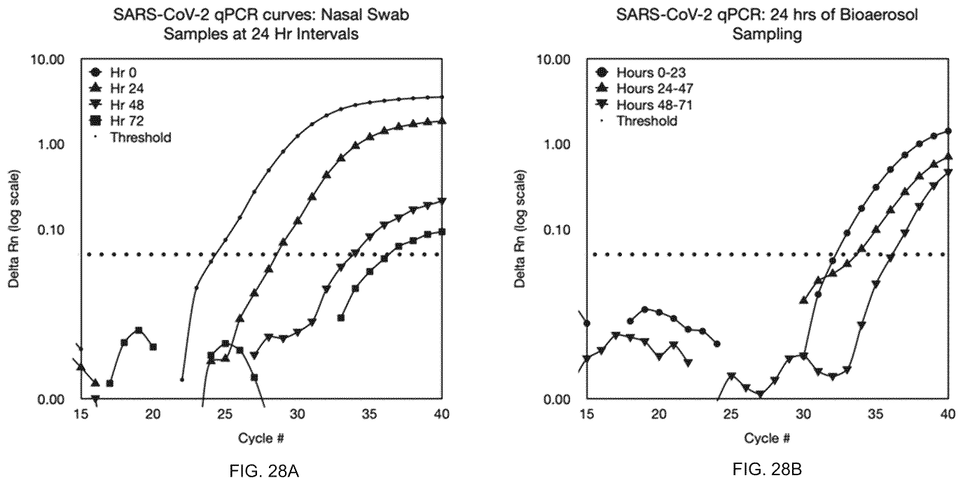

Air samples were taken from homes of persons known to be infected with SARS CoV-2. Nasal swab samples were taken as well. Both types of samples were taken at 24-hour intervals and demonstrate that air samples captured on a SCS, wherein the SCS containing analyte is directly amplified in an RT-qPCR reaction without an extraction or rinsing step, exhibit the same decreasing amount of virus detected as detected in nasal swabs of the infected individual. Thus, air sampling using the methods and compositions disclosed herein serve to accurately detect infected humans in an indoor environment with similar sensitivity as nasal swabs, but without the need for individual human testing, human consent, or even knowledge. FIG. 28A-FIG. 28B show two graphs each depicting SARS-CoV-2 qPCR curves. Each of these graphs depict Delta Rn (y-axis) vs. Cycle #(x-axis). Graph FIG. 28A shows SARS-CoV-2 qPCR curves for nasal swab samples at 24-hour intervals. To generate this data, human nasal swab samples were collected in a TRIS-EDTA buffer with SDS and 5 uL were directly added to 20 uL qPCR reactions using Fast-Virus MasterMix with additional Tween. Swabs were collected at 0 hours, 24 hours, 48 hours, and 72 hours. As seen in Graph FIG. 28A, delta Rn vs. cycle #decreases as time passes. Thus, delta Rn vs. cycle # is higher at hour 0, than at hour 24, than at hour 48, or than at hour 72.

Graph FIG. 28B shows SARS-CoV-2 qPCR curves ofbioaerosol sampling during the same time frame. The first of these time windows is from 0-23 hours, the second time window is from 24-47 hours, and the third time window is from 48-71 hours. To generate this data, bioaerosol was captured on SCS, and 3.0 mm punches of each SCS were placed directly into 50 uL PCR reactions. As seen in Graph FIG. 28B, the delta Rn vs. cycle #decreases time passes. Thus, delta Rn vs. cycle # is higher in the first time interval between 0-23 hours, than in the second or third time windows. RT-qPCR conditions were as follows: SC2FWD primer (5′-3′) CTGCAGATTTGGATGATTTCTCC (800 nM); SC2REV primer CCTTGTGTGGTCTGCATG AGTTTAG (800 nM); SC2probe/6FAM/ATTGCAACA/ZEN/ATC CATGAGCAGTGCTGACTC/3IBFQ/(800 nM). PCR program:10 mins 50° C. reverse transcription, 95° C. 20 seconds initial denaturation, (95° C. denaturation 3 seconds, 60° C. extension 30 seconds)×40 cycles.

### Example 2

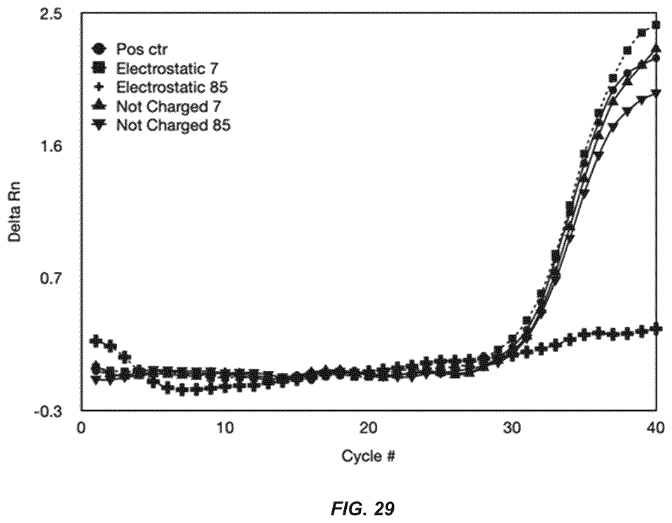

Candidate SCS materials were evaluated for compatibility with direct amplification in RT-qPCR reactions. FIG. 29 shows a graph depicting RT-qPCR curves for a positive control, and electrostratically charged material, (electrostatic KX polypropylene), and a non-electrostatically charged material (non-charged melt blown polypropylene). The graph depicts Delta Rn (y-axis) vs. Cycle #(x-axis). The graph includes two curves for each material, one of the curves showing 50 μL RT-qPCR reactions with approximately 7 mm² of the material, and the other of the curves showing RT-qPCR reactions with approximately 85 mm² of the material. Preferred SCS area is in the range of 50-200 mm². As seen, the electrostatically charged material significantly inhibits the RT-qPCR reaction as the amount of the electrostatically material is increased. Thus, in some embodiments, electrostatically charged material can be less suitable than non-electrostatically charged material for an SCS.

### Example 3

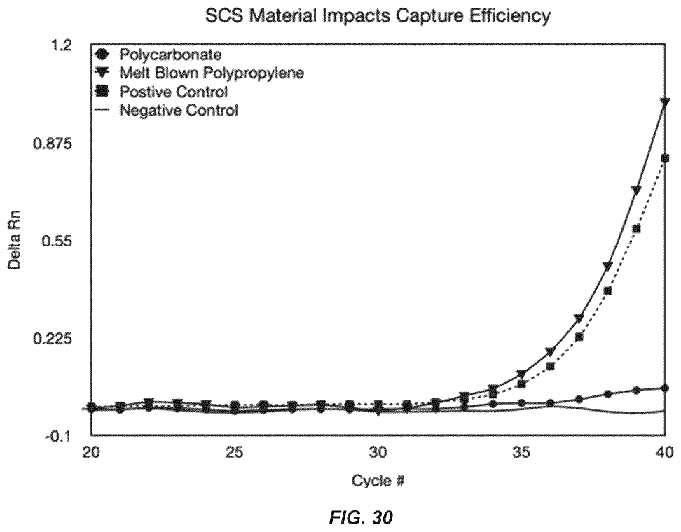

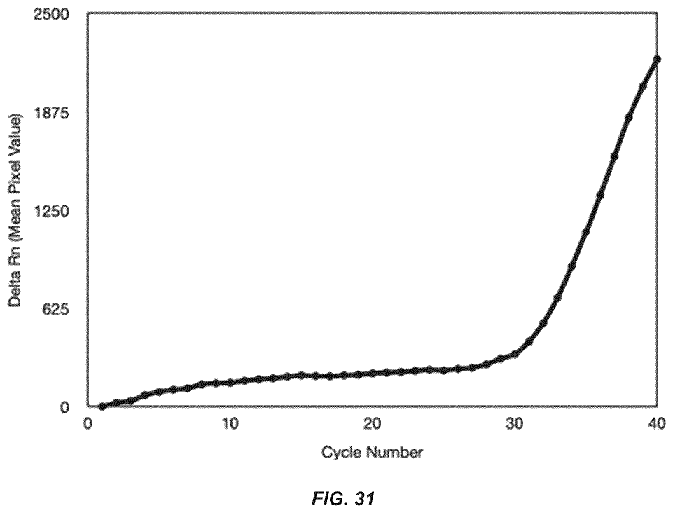

Candidate SCS materials were evaluated for efficiency of capturing aerosolized viral particles. FIG. 30 shows a graph depicting RT-qPCR curves for a positive control, negative control, polycarbonate (a commonly used air filter material) and melt-blown polypropylene. OC43 coronavirus was nebulized and then captured by pulling air through SCS materials at 50 L/min. 3 mm punches of SCS material were placed in 50 uL RT-pPCR reactions for amplification. Results demonstrate that not all materials used for air filtration and particle capture work effectively as SCS.

### Example 4

Example 4 shows capture and RT-qPCR amplification of aerosolized OC43 coronavirus using a cartridge body that includes a reaction chamber across which a non-electrostatically charged melt-blown polypropylene SCS is connected. OC43 viral culture fluid (Zeptometrics) at 10⁶ genome copies/uL was diluted 107 in phosphate buffered saline and nebulized inside a glove box. Resulting aerosols were captured on the SCS attached to the cartridge at a flow rate of 50 Lair per minute for 15 minutes. The SCS was then sealed inside the reaction chamber using sealing members. RT-qPCR reagents were added to the cartridge and conveyed into the reaction chamber through a fluidic channel in the cartridge. The cartridge was then processed in the thermocycling breadboard using a thermocycler and optics, with fluorescent images taken at the end of each extension step. Resulting images were processed by measuring pixel intensities using imageJ software. The initial mean pixel intensity was subtracted from subsequent cycle values to calculate a Delta Rn value. This end-to-end run shows the ability to capture virus, amplify and detect the nucleic acid directly from the SCS inside a reaction chamber in an integrated consumable cartridge.

In the foregoing specification, the invention is described with reference to specific embodiments thereof, but those skilled in the art will recognize that the invention is not limited thereto. Various features and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

1. An air sampling and analysis system, the system comprising:
a processor configured to execute stored computer instructions to control the air sampling and analysis system;
a consumable cartridge comprising a cartridge body and a sample capture substrate (SCS);
an air handler configured to move air through the SCS;
a thermocycler configured to bring the air sampling reaction chamber to one or more desired temperatures to perform direct nucleic acid amplification of nucleic acid sequences captured on the SCS; and
optics configured to measure fluorescent signal from the air sampling reaction chamber.

2. The system of claim 1, wherein the nucleic acids are amplified directly from the SCS, on the consumable cartridge, without a separate elution or purification step.

3. The system of claim 1, wherein the nucleic acid amplification occurs inside a chamber formed by sealing around the air sampling reaction chamber containing the SCS after the sampled air is moved through the SCS.

4. The system of claim 1, further comprising a cartridge receiver defining a receptacle configured to receive the SCS and at least a portion of the cartridge body, and wherein the SCS extends across an air sampling reaction chamber defined by the cartridge body; wherein the cartridge receiver comprises at least one sealing member sealingly couplable to the cartridge body around the air sampling reaction chamber to thereby enclose the air sampling reaction chamber and SCS.

5. The system of claim 3, wherein the cartridge body comprises a front and a back, wherein the sealing member comprises a first sealing member and a second sealing member, wherein the first sealing member is configured to sealingly couple to the front of the cartridge body around the air sampling reaction chamber, and wherein the second sealing member is configured to sealingly couple to the back of the cartridge body around the air sampling reaction chamber.

6. The system of claim 4, wherein the first sealing member is transparent, wherein the cartridge receiver is positioned with respect to the optics to expose the SCS to the optics through the first sealing member.

7. The system of claim 5, further comprising a sealer configured to seal the sealing member to the cartridge body.

8. The system of claim 1, further comprising: a mechanism for ejecting spent cartridge bodies and positioning new cartridge bodies for sample capture and analysis; and a magazine comprising a plurality of new cartridge bodies and cartridge receivers.

9. The system of claim 1, wherein the cartridge body comprises a first containment capsule comprising dry reagents and a second containment capsule comprising aqueous reagents; wherein the containment capsules are completely sealed, independently from the cartridge body.

10. The system of claim 9, wherein the cartridge body further comprises a first fluidic channel coupling the first containment capsule and the second containment capsule, and a second fluidic channel coupling the first containment capsule and the air sampling reaction chamber.

11. The system of claim 9, wherein the junction between one or more of the containment capsules and the cartridge can be opened without altering the volume of the containment capsules.

12. The system of claim 11, wherein the liquid and dried reagents can be mixed by alternately pressing the containment capsules to move the liquid.

13. The system of claim 12, further comprising one or more actuators to press the containment capsules in order to move the liquid.

14. The system of claim 1, wherein the cartridge functions are actuated at one or more separate positions inside a single instrument.

15. The system of claim 1, further comprising additional sensing components that signal air sampling when an associated analyte is detected or detected above a stated threshold value.

16. The system of claim 15, wherein the analyte is carbon dioxide.

17. The system of claim 1, wherein a surface of the reaction chamber contains at least one region containing nucleic acid capture probes that are bound to the surface and exposed to the nucleic acid amplification reaction; wherein the surface capture probes are homologous to at least one solution phase reporter nucleic acid; wherein the reporter nucleic acid binds to the surface bound capture probes in a manner proportional to the amount of a target nucleic acid captured on the SCS during air sampling.

18. A method of automated air sampling and analysis, the method comprising:
passing air through a sample capture substrate (SCS) to capture an analyte;
enclosing the SCS within a reaction chamber;
performing a direct nucleic acid amplification of nucleic acid sequences captured on the SCS within the reaction chamber; and
measuring a fluorescent signal from the reaction chamber and from the SCS, wherein the method is performed inside a single instrument without human intervention.

19. The method of claim 18, wherein performing the nucleic acid amplification reaction directly in the reaction chamber and on the SCS comprises: filling the reaction chamber with nucleic acid amplification reagents; and thermocycling the reaction chamber and the SCS.

20. The method of claim 19, wherein filling the reaction chamber with nucleic acid amplification reagents comprises: mixing aqueous nucleic acid amplification reagents with dry nucleic acid amplification reagents; and filling the mixed nucleic acid amplification reagents into the reaction chamber.
